(19) United States
(12) Patent Application Publication
(10) Pub. No.: US 2026/0297486 A1
(43) Pub. Date: Oct. 1, 2026

(54) 3D EPITHELIAL ORGANOID CULTURE SYSTEMS AND METHODS

(71) Applicant: The Regents of the University of California, Oakland, CA (US)

(72) Inventors: Hing Jii MEA, Davis, CA (US); Jiandi WAN, Davis, CA (US)

(21) Appl. No.: 19/480,630

(22) PCT Filed: May 31, 2024
(86) PCT No.: PCT/US2024/031924

§ 371 Date: Oct. 31, 2025

Related U.S. Application Data
(60) Provisional application No. 63/505,486, filed on Jun. 1, 2023.

(51) Int. Cl. C12M 1/12; C12N 5/00; C12N 5/071; G01N 33/50

(52) U.S. Cl. CPC C12M 25/14; C12N 5/0068; C12N 5/0679; G01N 33/5082; C12N 2513/00; C12N 2533/54; C12N 2533/74; C12N 2533/76; C12N 2533/90

(57) ABSTRACT

Disclosed herein are 3D culture systems and methods for producing epithelial tubular organoids, such as intestinal organoids. Also disclosed is a screening method that involves contacting elongated epithelial tubes produced from the disclosed systems and methods with a candidate agent, culturing the elongated epithelial tube in a growth medium under growth conditions, and evaluating the elongated epithelial tube for a physiological effect.

### CROSS-REFERENCE TO RELATED APPLICATIONS

This application claims benefit of U.S. Provisional Application No. 63/505,486, filed Jun. 1, 2023, which is hereby incorporated herein by reference in its entirety.

### BACKGROUND OF THE INVENTION

The gastrointestinal (GI) tract is not only responsible for digestive functions and absorption of nutrients, but also plays a key role in mediating the absorption or other bioactive compounds (e.g. drugs, gut flora metabolites) and shaping host immune responses (Lindell, A. E., et al. Nat. Rev. Microbiol. 2022 1-13; Ma, W. et al. Front. Microbiol. 2019 10). As such, it has come under increased scrutiny for potentially contributing to areas of human health far beyond the GI tissue itself. This has been demonstrated in the case of cardiovascular health (Wang, Z. et al. Nature 2011 472:57-65; Koeth, R. A. et al. Nat. Med. 2013 19:576-585; Gregory, J. C. et al. J. Biol. Chem. 2015 290:5647-5660; Claesson, M. J. et al. Proc. Natl. Acad. Sci. U.S.A. 2011 108:4586-4591; Jie, Z. et al. Nat. Commun. 2017 8:1-11) and in neurodegeneration (Holmqvist, S. et al. Acta Neuropathol. 2014 128:805-820; Scheperjans, F. et al. Mov. Disord. 2015 30:350-358; Braniste, V. et al. Sci. Transl. Med. 2014 6; Sampson, T. R. et al. Cell 2016 167:1469-1480.e12; Mertsalmi, T. H. et al. Eur. J. Neurol. 2017 24:1375-1383; Pietrucci, D. et al. Parkinsonism Relat. Disord. 2019 65:124-130), although the exact molecular mechanisms and causal links have yet to be studied in detail. While in vivo studies using animals such as gnotobiotic mice have been indispensable in observing associations between the gut and wider aspects of host health, in vitro models of the GI tract are highly coveted due to their relative simplicity and relative ease of use. Due to the absence of crosstalk and other convoluting factors associated with normal host physiology, simplified in vitro models of the gut epithelium have immense utility in facilitating molecular mechanistic studies that provide clinically-useful insights into the manifold ways in which the GI tract regulates human health. This is especially so considering the rising global health burden of not only GI-related chronic inflammatory conditions (e.g. inflammatory bowel disease, Crohn's disease and ulcerative colitis) (Kaplan, G. G. Nat. Rev. Gastroenterol. Hepatol. 2015 12:720-727; Li, K., et al. Front. Med. 2022 9:1-11), but also that of cardiovascular disease¹⁶ and neurological disorders (James, B. et al. Neurology 2014 82:1302; Winblad, B., et al. Lancet Neurol. 2016 15:455-532; Association, A. Alzheimer's Dement. 2019 15:321-387) and GI-related chronic inflammatory conditions.

Early in vitro models that use transformed cell lines such as Caco-2 or HCT116 have steadily been replaced by in vitro primary-derived intestinal epithelial tissues as the latter is demonstrably capable of producing models that successfully recapitulate physiological functions, model GI disease pathophysiology and studying drug responses (Sontheimer-Phelps, A. et al. Cmgh 2020 9:507-526; Jalili-Firoozinezhad, S. et al. Nat. Biomed. Eng. 2019 3; Co, J. Y., et al. Nat. Protoc. 2021 16:5171-5192). Models engineered from in vitro primary-derived intestinal epithelial tissues rely heavily on ECM materials such as MATRIGEL or collagen type I to enable successful cell proliferation and differentiation, whether as 3D organoids (Sato, T. et al. Nature 2009 459:262-265; Zhang, Y. G., et al. Physiol. Rep. 2014 2:1-11) or 2D surface monolayers (Wang, Y. et al. Cmgh 2017 4:165-182.e7; Wang, Y. et al. ACS Biomater. Sci. Eng. 2017 3:2502-2513; Nikolaev, M. et al. Nature 2020 585:574-578). Typically, Lgr5 stem cell-rich crypts are harvested from animals or patients and are then expanded in vitro to form organoids by suspending within ECM, typically MATRIGEL due to its similarity with basement membrane matrix, or plated on ECM substrates (e.g. collagen type I or MATRIGEL) to form a continuous surface monolayer. Nevertheless, these ECM materials possess extremely low stiffnesses (<1 kPa) and slow gelation kinetics especially in the case of collagen type I, which limit their ease of handling in bioengineering approaches. The same problem is more or less true for other recently developed scaffold materials derived from fibrin (Broguiere, N. et al. Adv. Mater. 2018 30) and polyethylene glycol (PEG) (Gjorevski, N. et al. Nature 2016 539:560-564) as these material systems were specifically engineered to match the low stiffness of MATRIGEL or native intestinal epithelium ECM.

### SUMMARY OF THE INVENTION

Disclosed herein is a 3D culture system that can produce epithelial tubular organoids. In some embodiments the 3D organoid culture system involves a filament having a bioactive hydrogel core and an inert hydrogel sheath, wherein the bioactive hydrogel core is an extracellular matrix hydrogel.

In some embodiments, the inert hydrogel has a diffusion rate for growth factors of from 1×10⁻⁷ to 9×10⁻⁷ cm²/s, including from 1×10⁻⁷ to 5×10⁻⁷ cm²/s, 5×10⁻⁷ to 9×10⁻⁷ cm²/s, 2×10⁻⁷ to 8×10⁻⁷ cm²/s, 3×10⁻⁷ to 7×10⁻⁷ cm²/s, 4×10⁻⁷ to 6×10⁻⁷ cm²/s, 3×10⁻⁷ to 9×10⁻⁷ cm²/s, and 1×10⁻⁷ to 7×10⁻⁷ cm²/s.

Without wishing to be bound by theory, the diffusion rate of the inert hydrogel can accentuate Wnt signaling in the transverse direction and result in hyperpolarization of the organoid while also allowing for a sufficient level of growth factors at the initiation of culture.

In some embodiments, the extracellular matrix hydrogel is a natural or synthetic hydrogel with active cell binding sites. In particular embodiments, the extracellular matrix hydrogel is MATRIGEL, collagen, or a mixture of MATRIGEL and collagen. For example, the extracellular matrix hydrogel can be a mixture of 90-70% (wt/v) MATRIGEL and 10-30% (wt/v) collagen.

In some embodiments, the inert hydrogel is polysaccharide hydrogel. For example, in some embodiments, the inert hydrogel is agarose or alginate.

In some embodiments, the core has a substantially cylindrical or cuboidal shape. In addition, but independently, the sheath can have a substantially cylindrical or cuboidal shape. Other shapes can be selected based on the choice of extrusion nozzles.

In some embodiments, the core has an average diameter or width of from 0.3 to 1.0 mm, including from 0.3 to 0.5 mm, 0.5 to 1.0 mm, 0.4 to 0.8 mm, 0.3 to 9.0 mm, 0.3 to 0.8 mm, 0.4 to 0.9 mm, or 0.4 to 1.0 mm.

In some embodiments, the sheath has an average thickness of from 2.0 to 6.0 mm, including 2.0 to 4.0 mm, 4.0 to 6.0 mm, 3.0 to 5.0 mm, 3.0 to 6.0 mm, or 2.0 to 5.0 mm.

In some embodiments, the bioactive hydrogel core does not comprise more than 0.05%, 0.1%, 0.2%, 0.3%, 0.4%, or 0.5% (wt/v) agarose. In some embodiments, the bioactive hydrogel core does not comprise any agarose.

In some embodiments, the filament comprises two or more bioactive hydrogel cores covered by the inert hydrogel sheath, e.g. 2, 3, 4, 5, 6, 7, 8, or more bioactive hydrogel cores covered by the inert hydrogel sheath.

In some embodiments, the system further involves stem cell organoids in the bioactive hydrogel core. For example, the core-sheath hydrogels can be produced using co-axial extrusion of a bioactive pre-gel solution and an inert pre-gel solution, wherein the organoids are present in a bioactive pre-gel solution prior to extrusion.

In some embodiments, the organoids are produced from stem cells or progenitor cells. For example, in some embodiments, the cells are intestinal, colonic, gastric, hepatic, pancreatic, rectal, mammary, or lung stem cells.

Also disclosed is a method for producing an elongated epithelial tube from stem cell organoids that involves culturing the disclosed 3D organoid culture system in a growth medium under growth conditions to produce the elongated epithelial tube.

In some embodiments, the stem cell organoid is an intestinal organoid and the growth medium is an intestinal organoid growth medium. Other combinations of organoid and growth medium are known in the art and can be used in the disclosed systems and methods. For example, in some embodiments, the organoid comprises duct epithelial cells, such as mammary or kidney duct epithelial cells.

In some embodiments, the method further involves removing the core containing the elongated epithelial tube from the extracellular matrix hydrogel.

Also disclosed herein is a screening method that involves contacting the elongated epithelial tube with a candidate agent, culturing the elongated epithelial tube in a growth medium under growth conditions, and evaluating the elongated epithelial tube for a physiological effect. For example, the physiological effect can be one or more biomarkers of differentiation, one or more stem cell biomarkers, one or more gene mutations that lead to inflammatory bowel disease or colon cancer, or any combination thereof.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1C illustrate various embodiments of a bioactive-inert hydrogel core-sheath structure for culturing epithelial organoids to form tubular organs. The bioactive hydrogel is shown in the core of a core-sheath structure. The sheath can be a cylinder (FIGS. 1A and 1B) or cuboid (FIGS. 1C and 1D). Likewise, the core can be a cylinder (FIGS. 1A and 1C) or cuboid (FIGS. 1B and 1D). Modulated diffusion of growth factors and other bioactive agents through inert sheath allows the organoids in the core to form a tubular structure.

FIGS. 2A to 2E show Agarose-Matrigel dual layers promote intestinal organoid growth and elongation in vitro. FIG. 2A is a schematic of the in vitro culture of intestinal organoids using agarose-Matrigel dual layers in a Transwell setup. Matrigel layer: 0.5 mm; Agarose layer: 3 mm, 1.5% w/v. FIG. 2B contains timelapse images of organoids grown in Matrigel only versus agarose-Matrigel dual layer in a Transwell setup. Scale bars: 250 μm. FIG. 2C contains plots of the degree of elongation in organoids (assessed by taking the ratio of the bud and core lengths) over the course of a 13-day culture. The solid line in the middle represents the median. FIG. 2D is a histogram of organoid bud lengths in Matrigel only versus agarose-Matrigel dual layer cultures at day 13. FIG. 2E contains immunofluorescence images of intestinal organoids with elongated morphologies. EdU was used to stain Lgr5 stem cells and transit amplifying cells undergoing proliferation and was pulsed for 24-hours in all the images. Actin was used to stain the entire cell cytoskeleton. Lysozyme was used to stain Paneth cells. The arrows in (iv) indicate regions stained positive for Lysozyme and are forming new budding structures. * p<0.05, ** p<0.01 and *** p<0.001, respectively, using Mann-Whitney U-test.

FIG. 3 shows bright-field and immunofluorescent staining of elongated organoids showing apical-in polarity of cells, with F-actin as the apical marker. Scale bar=250 μm.

FIGS. 4A to 4I show adjusting agarose layer changes the growth kinetics of intestinal organoids. FIG. 4A contains graphs comparing the 7-day organoid growth (projected area increase) in MATRIGEL-only versus dual layer cultures with 0.5 mm, 1.0 mm and 3.0 mm thicknesses of agarose layer. FIG. 4B is a graph comparing the bud lengths in MATRIGEL-only versus dual-layer cultures with 0.5 mm, 1.0 mm and 3.0 mm thicknesses of agarose layer. FIG. 4C contains histograms of bud length at day 7 for different agarose layer thicknesses. All agarose used in parts A-C are 1.5% w/v. FIG. 4D contains graphs comparing the 7-day organoid growth (projected area increase) in MATRIGEL-only versus dual layer cultures with 0.5, 1.5 and 3.0% w/v agarose. FIG. 4E is a graph comparing the bud lengths in MATRIGEL-only versus 0.5, 1.5 and 3.0% w/v dual-layer cultures. FIG. 4F contains histograms of bud length at day 7 for different agarose concentrations. All agarose used in parts D-F have a thickness of 1 mm. FIG. 4G contains graphs comparing the 7-day organoid growth (projected area increase) in dual layers with unconditioned agarose (prepared using PBS) versus conditioned agarose (prepared using growth media). FIG. 4H is a graph comparing the distribution of longest buds in MATRIGEL-only versus 0.5, 1.5 and 3.0% w/v dual-layer cultures. FIG. 4I contains histograms of longest bud length at day 7 for different agarose concentrations. All agarose used in parts G-I have a thickness of 3 mm and a concentration of 1.5% w/v. * p<0.05, ** p<0.01, *** p<0.001 and **** p<0.0001, respectively, using Mann-Whitney U-test. All violin plots show the median (solid line) and the 25^{th} & 75^{th} percentiles (dashed lines).

FIGS. 5A to 5F show transwell top compartment media composition influence organoid growth and prevalence of elongation phenomenon in intestinal organoids grown in agarose-MATRIGEL dual layers. All dual layers used have a thickness of 3 mm and a composition of 1.5% w/v. FIGS. 5A to 5C contain graphs comparing the 7-day organoid growth (projected area increase) and longest bud length when different top compartment media is used. Histogram shows the longest bud length distribution at day 7. FIGS. 5D to 5F contain graphs comparing the organoid growth (projected area increase) and longest bud length with different combinations of agarose conditioning and top compartment media composition. Histogram shows the longest bud length distribution at day 7. * p<0.05, ** p<0.01, *** p<0.001 and **** p<0.0001, respectively, using Mann-Whitney U-test. All violin plots show the median (solid line) and the 25^{th} & 75^{th} percentiles (dashed lines).

FIGS. 6A to 6E show Bioprinting MATRIGEL-agarose core-sheath filaments. FIG. 6A is a schematic of printing process for MATRIGEL-agarose core-sheath filaments. FIG. 6B contains images of MATRIGEL-agarose filaments. Part (i) shows spools of filament in a 60 mm diameter petri dish; part (ii) shows a 15 mm long filament with dense organoid suspension (~4000 organoids/mL) at day 12, with the right end sealed up with additional agarose post-printing; part (iii) shows the cross section of a MATRIGEL-agarose filament, with MATRIGEL outlined in blue; Blue arrows indicate the organoids inside MATRIGEL core. part (iv) shows micrographs of the organoids grown in part (ii). Scale bar is 500 μm. FIG. 6C contains timelapse images of organoids grown in different filaments. Interface between agarose and MATRIGEL shown in dotted lines. FIG. 6D contains violin plots of bud lengths for different MATRIGEL-agarose filaments. FIG. 6E is a histogram showing distribution of bud lengths at day 12. * p<0.05, ** p<0.01, *** p<0.001 and **** p<0.0001, respectively, using Mann-Whitney U-test. All violin plots show the median (solid line) and the 25^{th} & 75^{th} percentiles (dashed lines).

FIGS. 7A and 7B show heterogeneous growth of intestinal organoids in core-sheath filaments with thick cores (1.5 mm) over 12-day culture. FIG. 7A is a schematic of core-sheath filaments illustrating edge versus center regions of the filament. FIG. 7B contains bright-field timelapse images of organoids grown in MATRIGEL-agarose core sheath filaments.

FIGS. 8A to 8E show dislodged MATRIGEL filaments support intestinal organoid culture and 2D-3D hybrid morphologies. FIG. 8A contains a schematic and images of the MATRIGEL core dislodging process. Micrographs show the organoids in the MATRIGEL core at day 6 before and after dislodging. Scale bars are 5 mm and 250 μm for macroscopic images and micrographs, respectively. FIG. 8B contains violin plots comparing the bud lengths of organoids in filaments dislodged at different times. FIG. 8C contains histograms of bud lengths at day 12. FIG. 8D is a top-view of hybrid 2D-3D organoid morphologies on dislodged MATRIGEL filaments. Scale bars=50 μm unless otherwise noted. DAPI was used as nuclear counterstain; F-actin was used to mark the apical surface of cells that typically face inwards. Lysozyme was used to stain Paneth cells. FIG. 8E shows orthogonal views (front and cross-sectional) of 2D-3D organoid morphologies. Scale bars=50 μm * p<0.05, ** p<0.01, *** p<0.001 and **** p<0.0001, respectively, using Mann-Whitney U-test. All violin plots show the median (solid line) and the 25^{th} & 75^{th} percentiles (dashed lines).

FIG. 9A shows whole organoid showing 2D-3D hybrid morphology. Left: Bright field image of organoids. Center: Immunofluorescence image of intestinal epithelial with 2D-3D region highlighted. Right: 40× image of 2D-3D region. FIG. 9B is a 10× objective immunofluorescence image of intestinal epithelial tissue, with circled portions denoted areas with high expression of F-actin, suggesting surface breakthrough.

FIG. 10 is a schematic of dual layer effect leading to elongated organoids, with accumulation of Wnt antagonists leading to hyperpolarization of organoid to drive proliferation in the horizontal direction.

FIGS. 11A to 11D show adjusting agarose layer changes the growth kinetics of intestinal organoids. FIG. 11A is a graph comparing the bud to core length ratios in Matrigel-only versus dual-layer cultures with 0.5 mm, 1.0 mm and 3.0 mm thicknesses of agarose layer. FIG. 11B is a histogram comparing the bud length distribution at day 7 of culture using different agarose layer thicknesses. All agarose used in parts A-B are 1.5% w/v. FIG. 11C is a graph comparing the bud to core length ratios in Matrigel-only versus dual-layer cultures with 0.5, 1.5 and 3.0 w/v. % agarose concentration. FIG. 11C is a histogram comparing the bud length distribution at day 7 of culture using different agarose layer concentration. All agarose used in FIGS. 11C-11D are 1.0 mm thick. * p<0.05, ** p<0.01, *** p<0.001 and **** p<0.0001, respectively, using Mann-Whitney U-test. Statistically insignificant comparisons are not shown. All plots show the mean (solid line).

FIGS. 12A to 12D show bioprinting matrigel-agarose core-shell filaments. FIG. 12A is a schematic of printing process for Matrigel-agarose core-shell filaments. Part (i) shows spools of filament in a 60 mm diameter petri dish; part (ii) shows the cross section of a Matrigel-agarose filament, with Matrigel outlined; arrows indicate the organoids inside Matrigel core. Scar bar=500 μm. FIG. 12B contains timelapse images of organoids grown in filament. Interface between agarose and Matrigel shown in dotted lines. FIG. 12C is an image of an intestinal tissue from 3D printing. FIG. 12D contains confocal images of MINI gut. Scar bar for Part (i-iv)=500 μm. Scar bar for Part (v-viii)=250 μm.

FIGS. 13A and 13B show Agarose-Matrigel core-shell structure promotes muscle stem cells (MuSC) growth and differentiation in vitro. FIG. 13A is a bright-field image of MUSC differentiated myotube growth in agarose-Matrigel bioprinting filament over 7 days. Scale bar: 500 μm. FIG. 13B contains immunofluorescence images of Musc differentiated myotube with DAP, Actin, and myosin heavy train over 14 days. Scale bars: 500 μm.

FIGS. 14A and 14B show bioprinting agarose core-shell scaffold for MCF10A culture. FIG. 14A contains schematics of (i) printing agarose tube using a customized printhead nozzle and a glass capillary, (ii) injecting cell-containing bioink to printed agarose tube, (iii) a cross-section of bioink-filled agarose filament. FIG. 14B contains experimental images of (i) printed agarose tube, (ii) bioink filled agarose filaments, (iii) a zoom-in microscopic image of a bioink filled agarose filamen and (iv) its cross-sectional image. Dotted blue circle indicates the bioink and red arrow indicates MCF10A organoids. Bioink contains 40% (v/v) Matrigel and 10% (v/v) collagen. Images of ii-iv in B were taken after 4 days of culture in growth media.

FIGS. 15A and 15B show agarose core-shell scaffold promotes the growth of MCF10A into dense, connected mammary organoids. FIG. 15A contains brightfield images of the growth of MCF10A cells in (i) growth media only, (ii) growth media and differentiation media, and (iii) growth media, differentiation media and lactation media. Note differentiation media was added at day 6 in (ii) and lactation media was added at day 12 in (iii). FIG. 15B contains plots of normalized filament length over time. n=3, 6, and 14 filaments for growth media, growth and differentiation media, and growth, differentiation and lactation media, respectively. Dotted vertical lines dictate the day to change media type. Data are represented as mean±SD.

FIGS. 16A and 16B show laction media enhances the expression of epithelial mucin (MUC 1) in MCF10A organoids. FIG. 16A contains confocal images of the cross section of MCF10A organoids cultured in growth media only, growth and differentiation media, and growth, differentiation, and lactation at day 22 (left to right). DAPI stains for nucleus, MUC-1 stains for the mucin protein, Ep-CAM is an epithelial cell adhesion molecule for cell-cell contact. FIG. 16B contains qCR results.

FIGS. 17A to 17G show long-term culture creates transparent, hollow MCF10A organoids. FIGS. 17A and 17B contain brightfield images of the transparent MCF10A organoids cultured at 32 days at 5× magnification (FIGS. 17A) and 10× magnification (FIG. 17B), revealing a monolayer cell network on the organoid surface. FIGS. 17C and 17D contain confocal images of transparent mini-tissue stained with Ep-CAM, MUC-1, and DAPI at 63× magnification of the entire tissue (FIG. 17C) and its zoom image (FIG. 17D). FIGS. 17E and 17F show cross-sectional bright field (FIG. 17E) and confocal images (FIG. 17F) of the hollow MCF10A organoids. FIG. 17G shows organoid length over time for mini-tissues that were seeded as a monolayer with Matrigel and collagen into agarose, resulting in mini-tissue transparency at day 27.

FIGS. 18A to 18D show MCF10A cells grown with Matrigel into organoids, then seeded into agarose filaments with Matrigel and collagen. Organoids were cultured in the agarose filaments in growth media until Day 6, cultured in differentiation media until Day 12, and lactation media until Day 22 (FIG. 18A). FIG. 18B shows measured mini-tissue length over time. Organoids cultured with pure Matrigel cultured with all media types. MCF10A cells seeded with only Matrigel formed mini-tissues which aggregated on Day 3 and shrank in length over time until day 21 (FIG. 18C). FIG. 18D shows mini-tissue length over time of this culture grown with Matrigel only. No difference in mini-tissue growth and shrinkage between a bioink with pure Matrigel and a bioink with a mixture of collagen and Matrigel.

FIGS. 19A to 19G show transparent organoids grown by first culturing MCF10A cells in Matrigel for four days. Brightfield image of transparent organoid inside agarose at day 25 (FIG. 19A) and zoomed in to its monolayer surface (FIG. 19B). FIGS. 19C and 19D show confocal image of the same transparent organoid outside of agarose at 25 days (FIG. 19C) and zoomed in of the surface (FIG. 19D) Confocal images represent 100 stacked images. FIGS. 19E and 19F show confocal image (FIG. 19E) and brightfield (FIG. 19F) of cross section of transparent organoid at day 35. Confocal images represent 24 stacked images. FIG. 19G shows organoid length over time for mini-tissues that grew in Matrigel for 3 days prior to being seeded with Matrigel and collagen into agarose, resulting in mini-tissue transparency at day 25.

FIGS. 20A and 20B show timeline of transparent mini-tissues. Mini-tissue transparency is observed at Day 32 for cultures grown from MCF10A monolayer (FIG. 20A). Mini-tissue transparency is observed at Day 25 for cultures grown from MCF10A organoids cultured in Matrigel for 4 days (FIG. 20B).

FIG. 21 is a brightfield image of branched structure from popped organoid. This branching structure was one of the leaked contents of the transparent organoid after prodding the transparent organoid with a glass pulled needle. The branch is reminiscent of the tubular structures found in mammary glands, confirmed from other literature.

FIG. 22 is a brightfield image of MuSC differentiated myotube in parallel multi-channel agrose filament (channel number 5). Scale bar: 500 μm.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “hydrogel” refers to a matrix comprising a network of hydrophilic polymer chains. A “biocompatible hydrogel” is a polymer network that is not significantly toxic to living tissue and/or cells, and does not elicit an immunopathogenic response in healthy individuals. A biocompatible active mechanism is a process that is not toxic to particular cells or tissues, for example a temperature increase within the physiological temperature range of tissues, or that is applied briefly enough so as not to cause significant toxicity.

The term “culturing” cells refers to the process of keeping cells in conditions appropriate for maintenance and/or growth, where conditions refers to, for example, the temperature, nutrient availability, atmospheric CO₂ content and cell density in which the cells are kept. Cells can be cultured in vivo or in vitro. The appropriate culturing conditions for maintaining, proliferating, expanding and differentiating different types of epithelial cells are well-known and documented. The conditions suitable for organoid formation are those that facilitate or permit cell differentiation and the formation of multicellular structures.

The term “organoid” refers to three-dimensional culture systems of organ-specific cell types that develop from stem cells and self-organize (or self-pattern) through cell sorting and spatially restricted lineage commitment in a manner similar to the situation in vivo. An organoid therefore represents the native physiology of the cells [66] and is has a cellular composition (including both remaining stem cells, a near-physiological niche, as well as specialized cell types) and anatomy that emulate the native situation. Stem cells may be isolated from tissue or organoid fragments. The cells from which an organoid is generated differentiate to form an organ-like tissue exhibiting multiple cell types that self-organize to form a structure very similar to the organ in vivo. Organoids are therefore excellent models for studying human organs and human organ development in a system very similar to development in vivo. Epithelial cell organoids are organoids containing epithelial cells.

### 3D Organoid Culture System

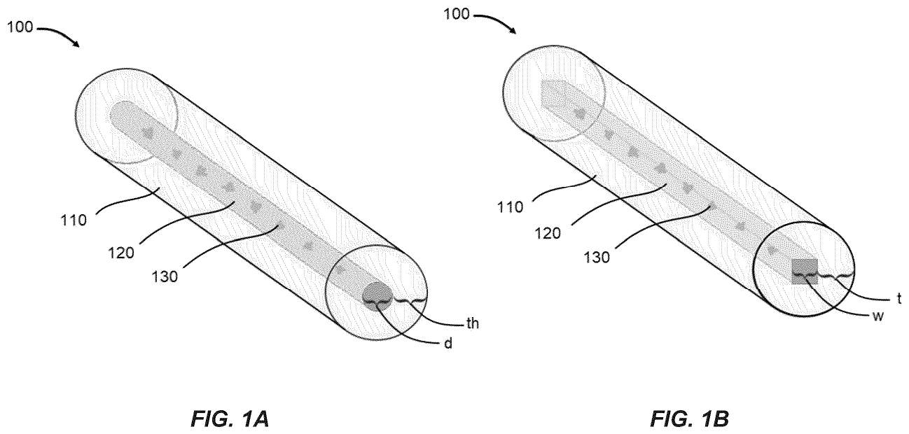

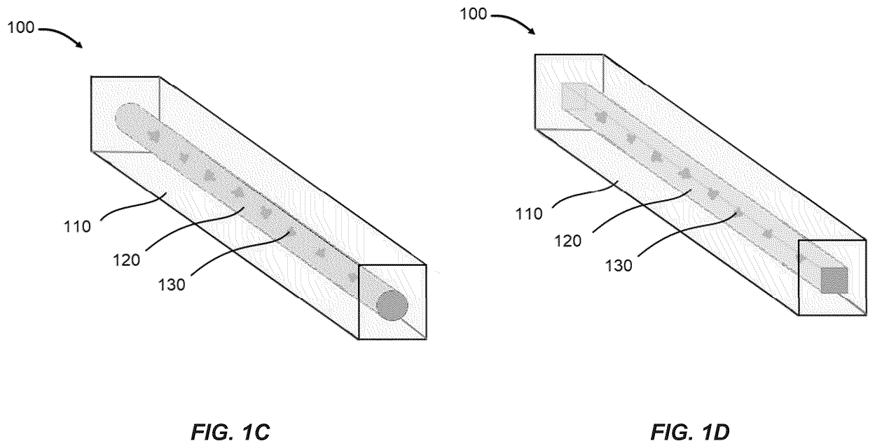

Turning now to FIGS. 1A to 1D, disclosed herein is a 3D organoid culture system 100 involving a filament having a hydrogel core 120 and an inert hydrogel sheath 110. As can be seen in FIGS. 1A to 1D, the core 120 and the sheath 110 can each independently have a substantially cylindrical or cuboidal shape. The core 120 can therefore have a length that is greater than its diameter (d) or width (w). The sheath 110 can also have a length that is greater than its thickness (th), i.e. the shortest distance from a position on the sheath's 110 outer surface to the core 120. Organoids 130 can be injected into the core 120 and cultured in a suitable growth medium.

In some embodiments, the bioactive hydrogel core 120 is an extracellular matrix (ECM) hydrogel. Examples, of ECM components include, but are not limited to, collagen, fibronectin, laminin, hyaluronates, elastin, and proteoglycans. For example, the multicellular aggregates, in some cases, contain various ECM proteins (e.g., gelatin, fibrinogen, fibrin, collagen, fibronectin, laminin, elastin, and/or proteoglycans).

In some embodiments, the bioactive hydrogel is derived from an ECM from an animal tissue or tumor. For example, the bioactive hydrogel can be derived from the basement membrane extracted from a tissue, such as a tumor. Examples include MATRIGEL, GELTREX, and LUNAGEL. In some embodiments, the bioactive hydrogel core 120 contains gelatin, collagen, fibrin, fibrinogen, laminin, fibronectin, protecogylcan, or any combination thereof.

In some embodiments, the bioactive hydrogel is a synthetic hydrogel with active cell binding sites. Synthetic hydrogels can be made from synthetic polymers, such as poly(acrylic acid) (PAA), poly(ethylene glycol) (PEG), poly(vinyl alcohol) (PVA), polyacrylamide (PAAm), and polypeptides.

In some embodiments, the core 120 contains at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (wt/v) MATRIGEL.

In some embodiments, the inert hydrogel is a polysaccharide hydrogel. Polysaccharides can be derived from plant, marine, animal and microbial metabolites. According to their main functional groups, natural polysaccharides utilized for hydrogel construction can be divided into anionic, cationic and neutral polysaccharides. In some embodiments, the inert hydrogel can be based on hyaluronic acid (HA), alginate, chitosan, dextran or cellulose. In some embodiments, the inert hydrogel is a starch.

In some embodiments, the inert hydrogel contains at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (wt/v) agarose and/or alginate.

In some embodiments, the organoids are produced from stem cells or progenitor cells. For example, in some embodiments, the cells are intestinal, colonic, gastric, hepatic, pancreatic, rectal, mammary, or lung stem cells or progenitor cells.

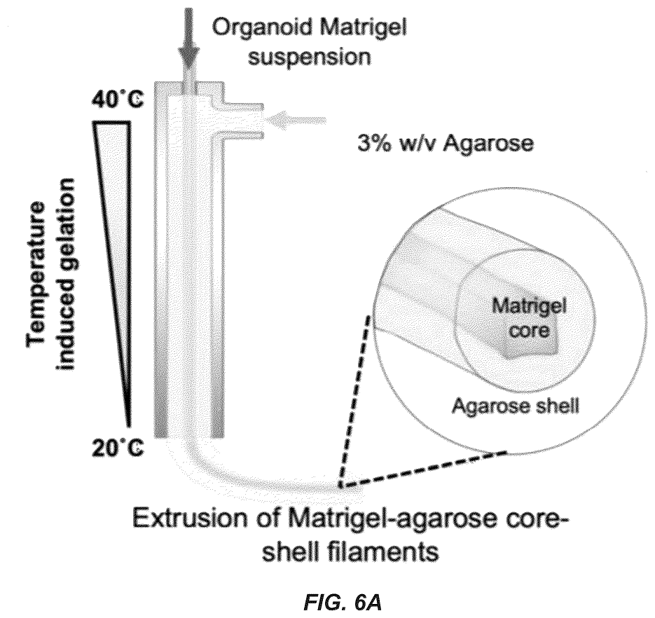

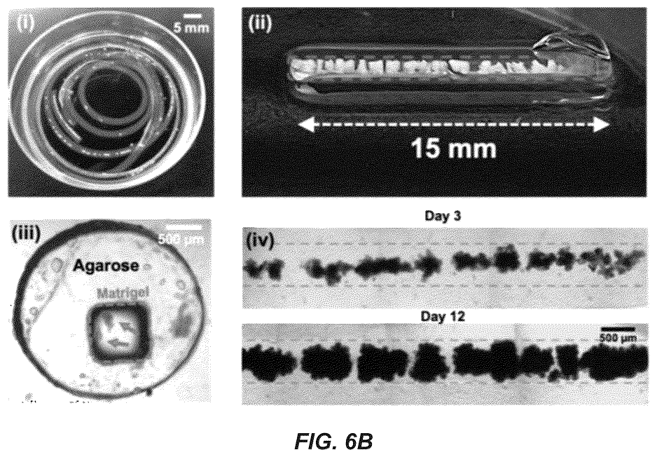

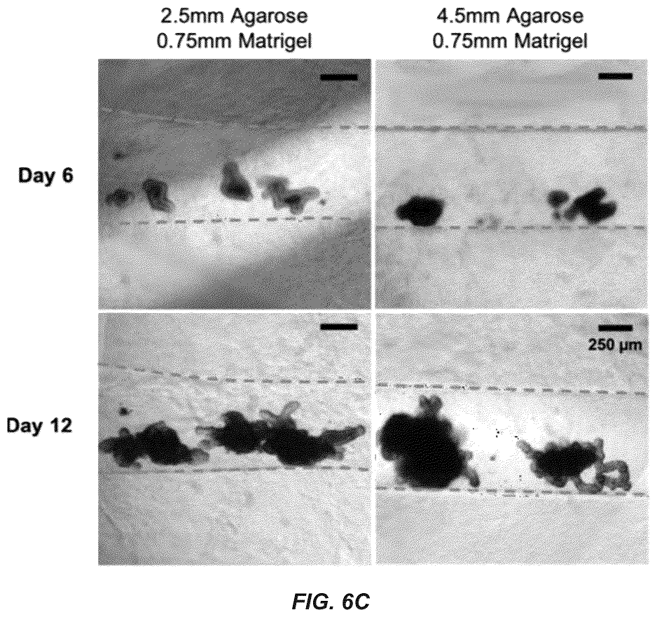

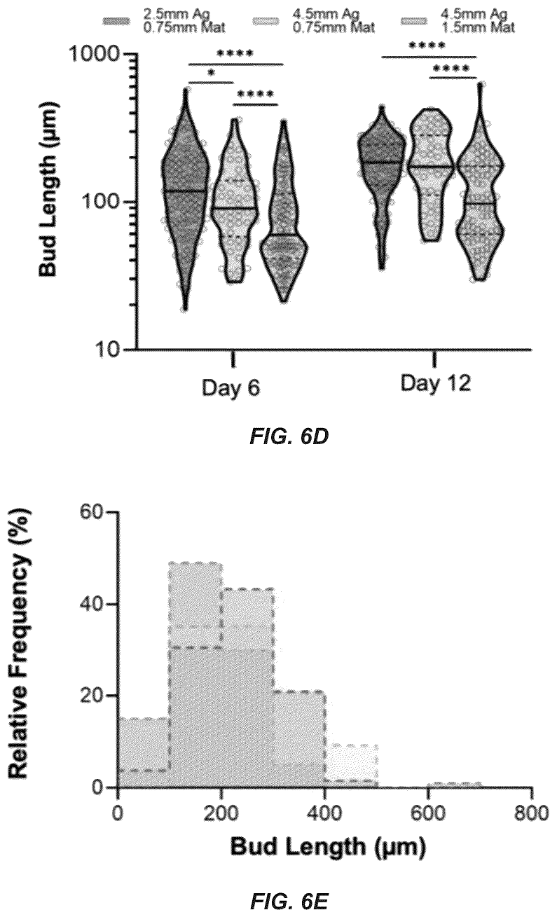

In some embodiments, the 3D organoid culture system disclosed herein can be produced using co-axial extrusion as shown in FIG. 6A. This process is well known in the art of 3D printing.

### Methods of Use

Disclosed herein is a method for producing elongated epithelial tubular structures, the method comprising encapsulating single stem cells or multicellular organoids in the core of the disclosed 3D organoid culture system and culturing the cells or organoids under suitable stem cell expansion conditions.

Also disclosed is a method for epithelial tissue regeneration comprising a) encapsulating and expanding of patient-derived epithelial stem cells or organoids in the disclosed 3D organoid culture system under suitable stem cell expansion conditions or suitable organoid formation conditions, and b) transplanting the expanded stem cells or organoids back into the patient.

Also disclosed is a method for screening of libraries of pharmacologic compounds, biomolecules or cells for physiological effects, the method comprising i) encapsulating tumor cells or organoids in the core of the disclosed 3D organoid culture system and culturing the cells or the organoids under suitable conditions in the presence of the compound or compounds to be tested, and ii) monitoring the elongated epithelial tubular structures.

In a further aspect, the invention provides a scalable and reproducible method for adapting the disclosed 3D organoid culture system for modeling human intestinal diseases such as cystic fibrosis (CF) and inflammatory bowel disease (IBD) in a manner that is amenable for personalized therapy. To this end, the disclosed 3D organoid culture system can be employed as screening tools to investigate the effect of pharmacologic compounds or biomolecules on in vitro grown intestinal biopsy samples from individual patients. Such a method for screening of libraries of pharmacologic compounds or biomolecules for efficacy in treating intestinal diseases, the method comprising i) providing intestinal biopsy sample from a patient, ii) encapsulating and growing the intestinal biopsy sample in the disclosed 3D organoid culture system and culturing the biopsy sample under suitable conditions in the presence of the pharmacologic compounds or biomolecules to be tested, and iii) in the case of cystic fibrosis, assessing the successful function restoration of the cystic fibrosis transmembrane conductance regulator (CFTR) by means of monitoring Forskolin-induced organoid swelling. iv) in the case of inflammatory bowel disease, monitoring the successful reduction in inflammation, cell damage or death, or restoration of epithelial junction integrity.

In a preferred embodiment, intestinal diseases are selected from the group comprising cystic fibrosis and inflammatory bowel disease.

The disclosed 3D organoid culture system are impactful as both basic and translational research tools. Intestine-specific processes, including ISC self-renewal, differentiation, crypt-villus patterning, inflammation and malignant transformation, as well as general epithelial phenomena, including establishment of apicobasal polarity and lumen formation, can be studied in a fully chemically defined and reproducible environment. The effects of microenvironmental parameters, including ECM proteins, cell-cell interaction proteins, matrix degradability and mechanical properties, on various aspects of intestinal biology can be elucidated. Furthermore, the chemically defined environment provided by the three-dimensional hydrogels of the disclosed 3D organoid culture system will be particularly valuable in the context of pharmacologic compound or biomolecules screens, costly large-scale endeavors where reproducibility and reliability are of utmost importance. Monolayers of the colorectal adenocarcinoma-derived Caco-2 cell line are the current norm as intestinal absorption models in pharmacokinetic studies of orally administered drugs. Primary intestinal organoids can serve as a histologically realistic complement or alternative to the Caco-2 model, also offering the potential for personalized studies using patient-derived organoids. Interfacing the hydrogel systems introduced here with robotic and liquid-handling technologies to afford high-throughput drug screening is readily conceivable. The disclosed 3D organoid culture system can be readily adapted for expanding and transplanting not only ISCs and intestinal organoids but also other types of epithelial stem cells and organoids, including those derived from the stomach, colon, rectum, pancreas, liver, lung and mammary gland.

### Embodiments

Embodiment 1. A 3D organoid culture system comprising a filament having a bioactive hydrogel core and an inert hydrogel sheath, wherein the bioactive hydrogel core is an extracellular matrix hydrogel, and wherein the inert hydrogel has a diffusion rate for growth factors of from 1×10⁻⁷ to 9×10⁻⁷ cm²/s.

Embodiment 2. The system of embodiment 1, wherein the extracellular matrix hydrogel is MATRIGEL or collagen.

Embodiment 3. The system of embodiment 2, wherein the extracellular matrix hydrogel is a mixture of MATRIGEL and collagen.

Embodiment 4. The system of embodiment 3, wherein the extracellular matrix hydrogel comprises 90-70% (wt/v) MATRIGEL and 10-30% (wt/v) collagen.

Embodiment 5. The system of any one of embodiments 1 to 3, wherein the inert hydrogel is polysaccharide hydrogel.

Embodiment 6. The system of embodiment 5, wherein the inert hydrogel comprises agarose or alginate.

Embodiment 7. The system of any one of embodiments 1 to 6, wherein the core has a substantially cylindrical or cuboidal shape.

Embodiment 8. The system of any one of embodiments 1 to 7, wherein the sheath has a substantially cylindrical or cuboidal shape.

Embodiment 9. The system of any one of embodiments 1 to 8, wherein the core has an average diameter or width of from 0.3 to 1.0 mm.

Embodiment 10. The system of any one of embodiments 1 to 9, wherein the sheath has an average thickness of from 2.0 to 6.0 mm.

Embodiment 11. The system of any one of embodiments 1 to 10, wherein the bioactive hydrogel core does not comprise more than 0.1% (wt/v) agarose.

Embodiment 12. The system of embodiment 11, wherein the bioactive hydrogel core does not comprise agarose.

Embodiment 13. The system of any one of embodiments 1 to 12, further comprising stem cell organoids in the bioactive hydrogel core.

Embodiment 14. A method for producing an elongated epithelial tube from stem cell organoids comprising (a) providing the system of embodiment 13; and (b) culturing the system in a growth medium under growth conditions to produce the elongated epithelial tube.

Embodiment 15. The method of embodiment 14, wherein the stem cell organoid is an intestinal organoid and wherein the growth medium is an intestinal organoid growth medium.

Embodiment 16. The method of embodiment 14 or 15, further comprising removing the core containing the elongated epithelial tube from the extracellular matrix hydrogel.

Embodiment 17. An elongated epithelial tube produced by the method of any one of embodiments 14 to 16.

Embodiment 18. A screening method comprising contacting the elongated epithelial tube of embodiment 17 with a candidate agent, culturing the elongated epithelial tube in a growth medium under growth conditions, and evaluating the elongated epithelial tube for a physiological effect.

Embodiment 19. The method of embodiment 18, wherein the physiological effect comprises one or more biomarkers of differentiation, one or more stem cell biomarkers, one or more gene mutations that lead to inflammatory bowel disease or colon cancer, or any combination thereof.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Bioprinting Multi-Layer Hydrogels for Intestinal Epithelial Tissue Engineering

Here, the use of agarose in conjunction with MATRIGEL was explored for in vitro intestinal epithelial engineering. By stacking layers of MATRIGEL and agarose (which we coin “dual layers”), it was possible to support the growth of intestinal organoids in vitro. Analysis of organoid growth and morphology using bright field and immunofluorescence microscopy reveal the presence of highly elongated organoids grown on dual layers marked by extended proliferative budding structures similar to intestinal crypts in vivo. Using these findings, a protocol is also reported for bioprinting core-sheath filaments consisting of MATRIGEL and agarose to produce in vitro intestinal epithelial tubes that are easy to handle due to the mechanical integrity of agarose. This protocol was also used to generate thin sub-millimeter diameter MATRIGEL filaments suspended with organoids that are not only capable of producing elongated organoids, but also hybrid 2D-3D intestinal epithelial tissue morphologies that exhibit apical-out epithelial monolayers as well as crypt structures.

### Materials and Methods

### Reagents:

DMEM/F12 with HEPES, IntestiCult™ Organoid Growth Media (mouse), Mouse intestinal organoids and Gentle Cell Dissociation Reagent were purchased from StemCell Technologies. 30 wt. % BSA solution was purchased from VWR. MATRIGEL, growth factor-reduced MATRIGEL (#356231) and Costar 96-well plates (#3595) were purchased from Corning. UltraPure™ low melting point agarose (#16520100) was purchased from Invitrogen.

### Intestinal Organoid Culture and Expansion:

Mouse intestinal organoids were purchased from StemCell Technologies in cryovials. To thaw the organoids from cryopreservation, the vials were warmed in a water bath at 37° C. until the contents of the cryovial have mostly liquefied. The contents were promptly mixed with 5 mL of 1 wt. % BSA solution in DMEM and centrifuged at 200×RCF for five minutes. The organoids were then resuspended in 30 μL of 1:1 ratio of MATRIGEL and INTESTICULT media per well, pipetted onto 4 wells within 24-well tissue culture plates, and incubated at 37° C. for 10 minutes to allow polymerization of MATRIGEL. 700 μL of IntestiCult is then added into the well, and the media was exchanged every two days.

Wells were selected for subculturing upon organoids exhibiting pronounced budding/crypt-like structures (typically within 5-9 days). Media was aspirated before introducing 1 mL of cold Gentle Cell Dissociation Reagent into these wells. The contents of the wells were then pipetted up and down 20 times to break up the MATRIGEL before being transferred into a 15 mL conical tube. The tube containing the resuspended organoids were then mechanically dissociated further by inverting for 10 minutes before being centrifuged at 300×RCF for five minutes. The supernatant was discarded before resuspending the organoids in 10 mL of DMEM/F12 and centrifuging at 200×RCF for five minutes. All centrifugation steps were performed at 4° C. The supernatant was then discarded, leaving behind a pellet consisting of dissociated organoids that could be sub-cultured for subsequent regular organoid culture maintenance or for experiments.

### Hydrogel Preparation and Organoid Growth Experiments in Transwell Inserts

Agarose solution was prepared by dissolving the appropriate weight percent of agarose into 1×PBS. In order to ensure the osmotic pressures are in the expected range when constituting 1×PBS agarose, DI water was added to replenish any volume lost due to evaporation. For agarose conditioning, agarose pre-gel solution was mixed at a 1:1 ratio with IntestiCult media to form a final agarose concentration of 1.5 w/v % agarose. Once the agarose solution is properly constituted, it was added into the Transwell insert at a volume corresponding to a desired layer thickness (~32 μL per mm thickness) and allowed to undergo gelation for 10 minutes at room temperature.

Following this, organoid-laden MATRIGEL suspension was prepared by mixing MATRIGEL in a 1:1 ratio with organoid-laden (~800 organoids per mL) IntestiCult media prior to being pipetted onto the Transwell insert for a 24-well plate. A 0.5 mm-thick layer was formed in the Transwell insert by pipetting the appropriate volume of the MATRIGEL mixture either on top of the agarose or on the bare Transwell, and the MATRIGEL was then allowed to undergo gelation for 10 minutes at 37° C. Following this, 700 μL of IntestiCult growth media was added to the bottom compartment while 100 μL of IntestiCult growth medium was added to the top (Transwell) compartment, thus completing the setup. The media in the bottom compartment was replaced every 2 days, while the top compartment was left untouched throughout the culture to avoid washing away the organoids suspended in MATRIGEL.

### Growth Quantification

Images were taken using a Leica DMi8 microscope, and all images were analyzed using ImageJ. Growth was assessed by measuring the projected area of the organoids in select locations in each well over the course of the culture. The measured areas from days 3, 5, 7, 9, 11 and 13 were normalized by the projected area of organoids on day 1. Organoid longest bud lengths at various days were determined by tracing the longest budding structure of each organoid through its center to where it meets the darkened rounded core of the organoid. In the case where organoids displayed a high degree of blebbing and darkening to the point where no viable budding structures could be identified, longest bud length was determined by measuring the length of any darkened structures that resembled typical budding structures.

### Immunofluorescent Staining:

Solutions prepared for use in immunofluorescent staining include 10% Formalin, permeabilization/washing solution (0.2% Triton X-100+0.05% Tween-20 in PBS), blocking solution (5% goat serum+5% BSA in washing solution), primary antibody solution (rabbit anti-mouse Lysozyme monoclonal antibody, MA5-32154, 1:50 dilution in blocking solution) and secondary antibody solution (anti-rabbit goat Alexa Fluor 594 secondary antibody, A32740, 1:500 dilution in blocking solution).

Prior to fixing, organoids were suspended in cold PBS for up to 10 minutes to ensure dissociation of MATRIGEL. Organoids were then fixed and permeabilized using 10% Formalin for 20 minutes and washing solution for 20 minutes, respectively. Following this, organoids were blocked with 5% goat serum+5% BSA in washing solution (0.2 % Triton X-100+0.05% Tween-20 in PBS) overnight at 4° C. Next, organoids were incubated with primary antibodies solution overnight at 4° C., followed by washing 3 times with washing solution. Following this, secondary antibody solution was added to the cells for 1 hour at room temperature, followed by 3 more washing steps. Finally, Hoechst 33342 and optional phalloidin stain was added to the cells. Organoids were then transferred to a glass chamber slide for subsequent confocal imaging. For EdU staining, Click-iT EdU imaging kits were used following the manufacturer's protocols.

### Core-Sheath Filament Printing Setup

A coaxial printhead assembly was built using a glass capillary (side length of 0.4 or 1.05 mm), a male-to-male luer connector and a custom 3D-printed nozzle (outlet diameter of 2.5 or 4.5 mm). The 3D-printed nozzle was designed on SolidWorks and made using Formlabs Form 3B SLA printer with Surgical Guide resin. The glass capillary was centered inside the barrel of the 3D-printed nozzle prior to being glued to a male-to-male luer connector to form a watertight seal and complete the coaxial printhead assembly. Polyethylene tubing (16 or 27.5 Birmingham needle gauge) was used to connect the coaxial printhead assembly to the bioink feeds.

Agarose (3% w/v) in the liquified pre-gel state was added to a 10-mL syringe. The syringe was subsequently wrapped in a syringe heating jacket (New Era Pump Systems) set at 40° C. and loaded onto a syringe pump. The syringe was connected via 16-gauge polyethylene tubing to the 3D-printed nozzle and the tubing was quickly filled with agarose prior to setting the flow rate at between 0.3 to 0.4 mL/min to produce filaments. Upon verification of steady-state agarose filament production, organoids-laden MATRIGEL suspension was added into a 1-mL syringe which was then loaded onto a separate syringe pump whose flow rate was set to match the velocity of the agarose layer. Core-sheath agarose filaments were collected in a 60mm-wide Petri dish and at 37° C. for 10 minutes upon completion of printing. Immediately prior to adding 6 mL media for 12-day culture, the filaments were optionally sealed with 3% w/v. agarose at the ends to prevent unintended dislodging of the MATRIGEL core mid-culture. To trigger dislodging of MATRIGEL core, ends were cut/left open and media was lightly perfused to impinge upon the MATRIGEL core until it slides out. All filaments/cores were cultured in 6 mL of IntestiCult media for 12 days, with 3 mL of the media being exchanged once at day 6.

### Statistical Analysis

Statistically significant differences in organoid projected area and longest axis were assessed using nonparametric Mann-Whitney U-test in GraphPad Prism when p-values under 0.05. Annotations used include: ns=not significant, *=p<0.05, ** =p<0.01, *** =p<10⁻³, **** p<10⁻⁴.

### Results

### Elongated Organoids on Agarose-MATRIGEL Dual Layers

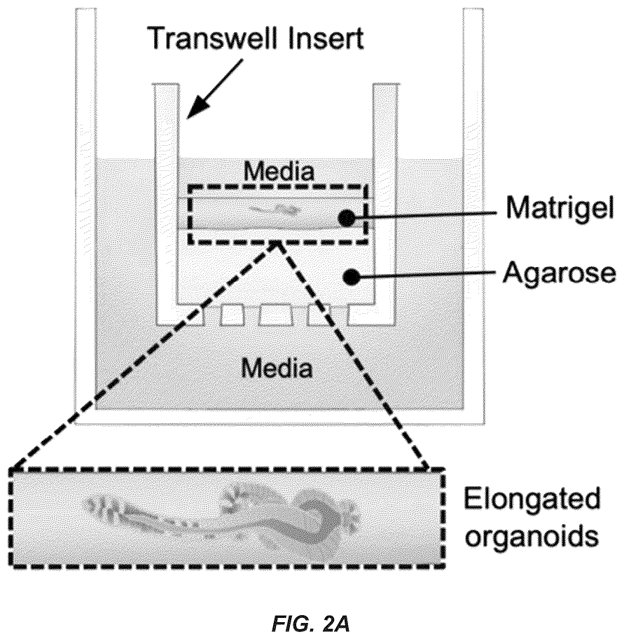

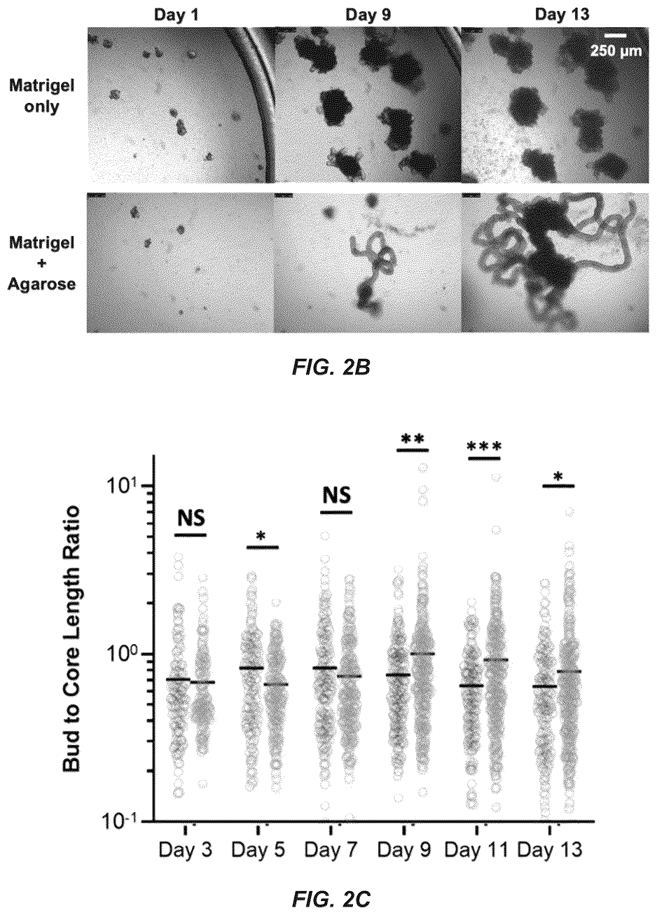

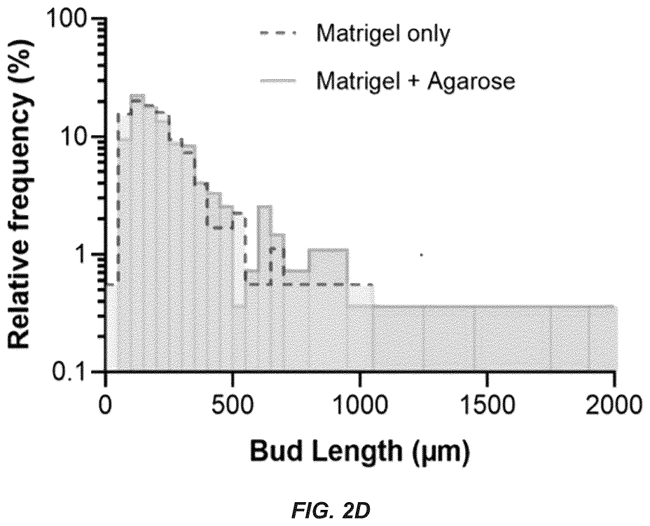

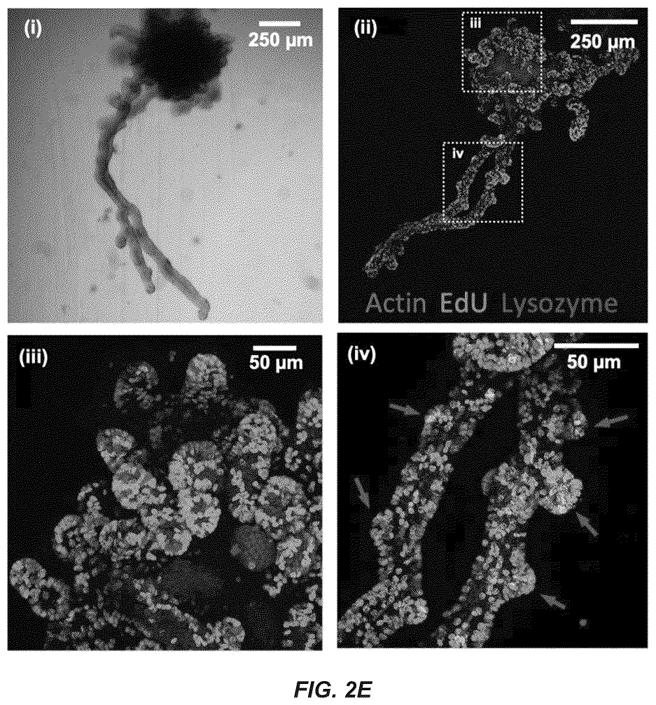

To test the compatibility of agarose with MATRIGEL, a Transwell insert setup was used to compare the growth and expansion of murine intestinal organoids when cultured in MATRIGEL versus a dual layer setup featuring a layer of agarose atop the organoid-laden MATRIGEL layer (FIG. 2A). Since, agarose is water-permeable and allows the transport of nutrients and growth factors much like the track-etched membrane on the bottom of the Transwell insert, it was hypothesized that dual layers should support cell/tissue culture much the same its MATRIGEL counterpart, so long as agarose is constituted to resemble physiological osmolality. Timelapse imaging over 13-days of culture revealed that both MATRIGEL-only and dual layer trials exhibit standard intestinal organoids growth behavior. Specifically, organoids initially produce Lgr5 stem cell-rich budding structures and gradually develop darkened cores that then overtake the budding structures (Sato, T. et al. Nature 2009 459:262-265). By day 13, many of the organoids grown in the MATRIGEL-only control exhibited a low number of buds and predominantly consisted of the darkened cores. At the same time, within the dual layer experiments, there was a subset of organoids where one or several budding structures grew far beyond (>500 μm) the bounds of the darkened core, resulting in a highly elongated and snaking appearance in contrast to the more rounded appearance of regular organoids (FIG. 2B). Overtime, these highly elongated buds also begin to round out and form darkened cores, as seen in the day 7 and 13 images.

Analyzing the bright field timelapse images, the growth kinetics of dual layer organoid cultures shows broad similarity with the MATRIGEL-only control at the start of the 13-day culture. Starting at day 9, however, the median fold-increase of the dual layer setup begins to overtake that of the MATRIGEL-only control. By day 13, median fold-increase was 34.4-fold (n=22) vs 16.5-fold (n=23) in the dual layer versus MATRIGEL-only control, respectively (FIG. 2C). The same trend is present when analyzing the longest organoid bud length. In both dual layers and MATRIGEL-only controls, the bud length increases steadily and peaks at day 11, before falling off at day 13. Dual layers generate comparatively shorter buds compared to the MATRIGEL-only controls up to day 9 but overtake the MATRIGEL-only experiments starting in day 11, with the median longest bud length in dual layers exceeding that of the MATRIGEL-only experiments by 34% and 91% at days 11 and 13, respectively. This is further evidenced by the distribution of longest bud lengths at day 11, with 25.6% organoids in dual layers longest bud lengths being greater than 500 μm compared to 12.5% in MATRIGEL-only (FIG. 2D). Altogether, these results indicate that addition of the agarose layer in the Transwell setup is not only compatible with MATRIGEL for intestinal organoid culture but helps to induce highly proliferative organoid phenotypes that can outperform MATRIGEL-only cultures.

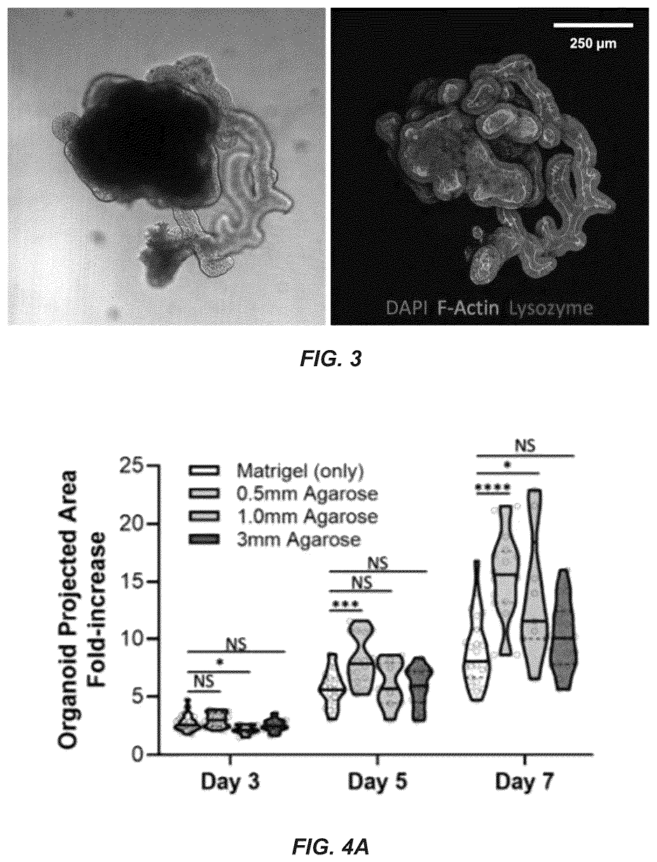

Immunofluorescence staining revealed that the highly elongated budding structures contain a high proportion of EdU positive cells (after a 24-hour pulse) and a lesser number of Paneth cells localized in regions where new budded structures are emerging as evidenced by the curvature (FIG. 2E). In terms of apical-basal polarity, F-actin staining shows the apical surface facing the internal volume of the organoid, consistent with the apical-in polarity of regular intestinal organoids (FIG. 3). These results indicate that the highly elongated budding structures are reminiscent of regular Lgr5 stem cell-rich budding structures seen in regular intestinal organoids. Their elongated appearance compared to regular budding structures is due primarily to their relatively high level of proliferation. At the same time, the presence of Paneth cells along the long budding structure is likely indicative of the production of new Lgr5 stem cell-rich budding structures, similar to crypt fission events in vivo that lead to formation of new crypts (Langlands, A. J. et al. PLoS Biol. 2016 14:1-31).

### Agarose Layer Properties Directly Influence Intestinal Organoid Growth in Dual Layers

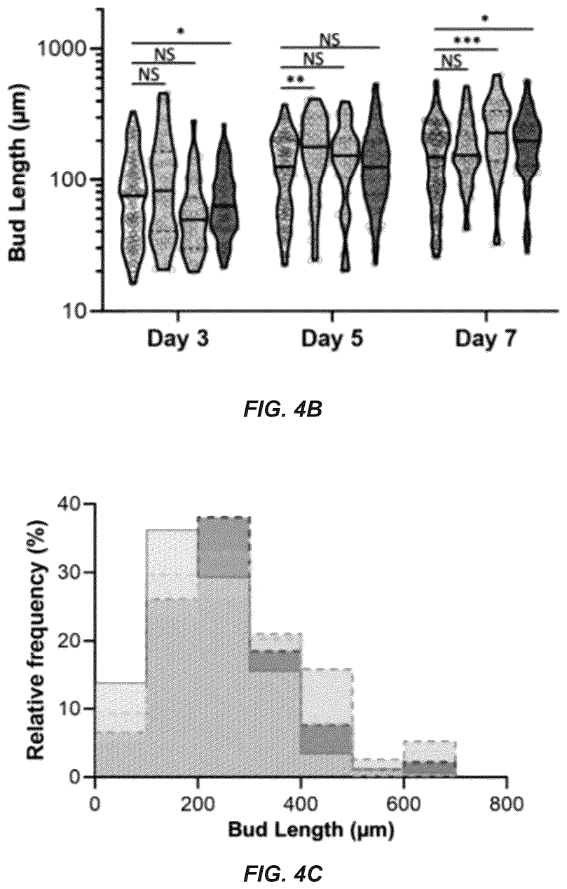

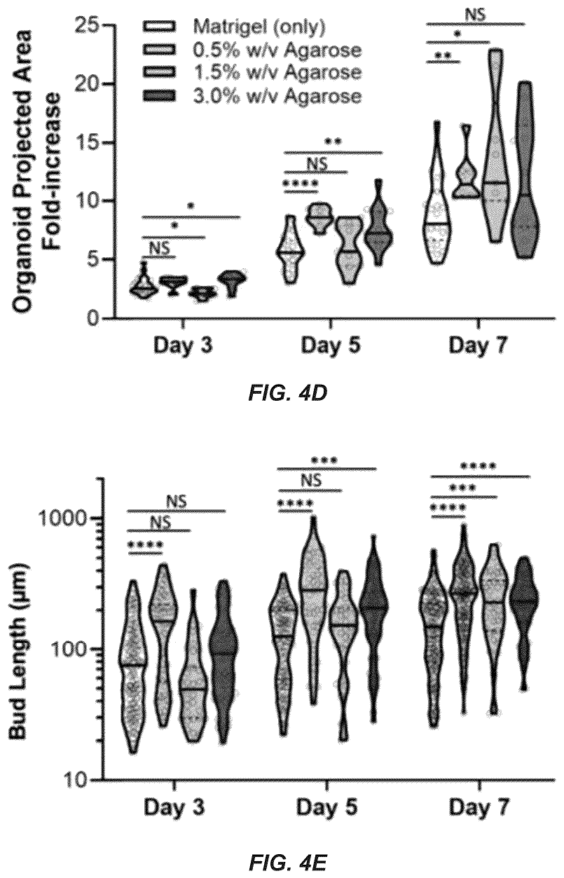

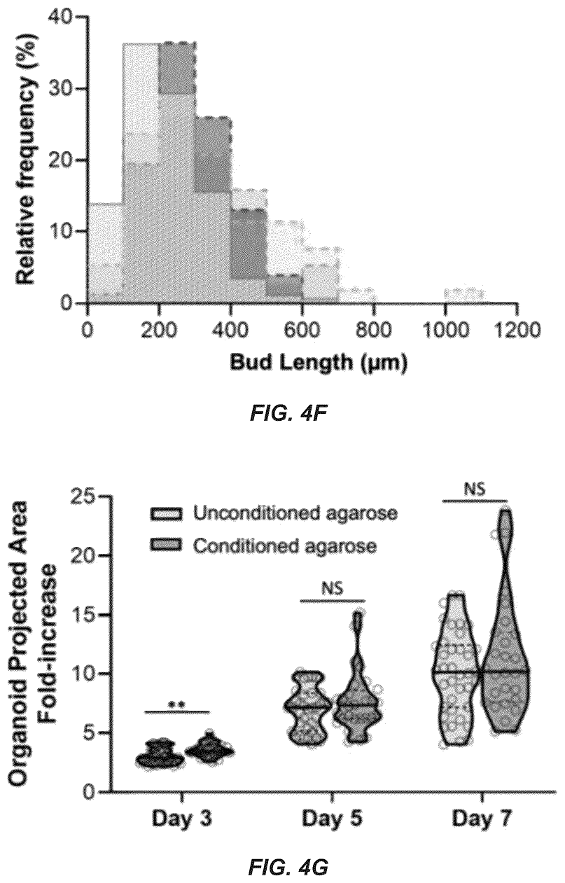

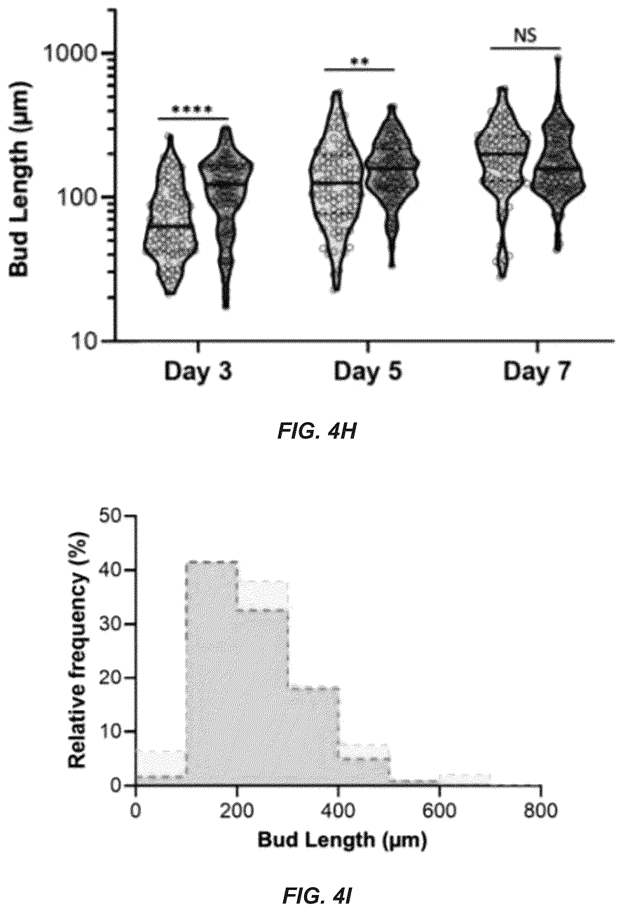

To elucidate the means by which the addition of agarose in the dual layer organoid culture promotes elongated morphology and organoid growth, the Transwell growth experiments were repeated while making changes in agarose thickness and concentration. Analysis of organoid growth revealed that thinner agarose layers provide a significant improvement as early as day 3 in the case of 0.5 mm. At day 7, the 0.5 mm dual layer had produced a median fold-change of 15.61 (n=16) versus 8.05 (n=11) from the MATRIGEL-only control. This is particularly striking considering how parity with the MATRIGEL-only organoid culture was only achieved by the dual layer in day 9 as shown in FIG. 2 when the agarose layer was 3 mm. Similarly, addition of thicker agarose layers (1.0 and 3.0 mm) also results in an improvement in organoid growth over the MATRIGEL-only culture, but only at later days (i.e. day 7) (FIG. 4A). When comparing the distribution of longest bud lengths, a similar trend was observed with the thinnest agarose layer giving a comparatively early boost to longest bud length at day 5, in contrast to thicker agarose layers which did so at day 7. The 1.0 mm agarose layer produced the highest median longest bud length of 229.7 μm, which is 29.3% higher than 177.7 μm from MATRIGEL-only experiments (FIG. 4B). Comparing the histograms of longest bud lengths at day 7 reveals a consistent right shift when agarose layer is added (FIG. 4C).

Changing the agarose permeability had a similar effect to with varying the thickness. When agarose concentration was decreased from 1.5 to 0.5% w/v, it conferred an early benefit to the organoid growth; quantitatively, the 0.5% w/v produced a 3.15-fold change in organoid projected area versus 2.57-fold from the control at day 3. Strikingly, an early improvement of similar magnitude (3.37-fold) was also seen when increasing the agarose concentration from 1.5 to 3.0% w/v. In subsequent days, however, the early improvement from 3.0% w/v agarose over the MATRIGEL-only culture all but disappeared, while lower agarose concentrations produced a sustained improvement in organoid growth throughout the 7-day culture (FIG. 4D). A similar trend is seen when analyzing the longest bud length where the 0.5% w/v agarose provided an early and sustained improvement, reaching a maximum median longest bud length of 267.6 μm at day 7 compared to 232.2 μm from the 3.0% w/v dual layers (FIG. 4E). Comparing the distribution of longest bud lengths show that 0.5% w/v agarose has a clear lead over the more concentrated (or less permeable) agarose dual layers (FIG. 4F). Taken together, thinner and more permeable agarose layers confer an earlier benefit to organoid growth than thicker and less permeable layers. This may be attributed to earlier breakthrough times for the diffusion of growth factors/nutrients to the organoid-laden MATRIGEL layer.

Next, it was hypothesized that the initial concentration of growth factors/nutrients in the proximity of the organoids in the MATRIGEL layer plays a critical role in the successful growth of organoids in dual layers. To produce a higher initial concentration, the agarose layer was conditioned by mixing it with intestinal organoid growth media at a 1:1 ratio prior to plating. The Transwell experiments were then repeated comparing regular dual layers (or unconditioned) with conditioned dual layers over a 7-day culture.

Organoid projected area measurements revealed a boost conferred by conditioning agarose as early in day 3 (3.45-fold versus 2.91-fold), and that this boost effectively disappears in subsequent days (FIG. 4G). A similar trend is seen in the longest bud length data where the conditioned agarose dual layers saw an improvement over its unconditioned counterpart between days 3-5, but no advantage by day 7 (FIG. 4H), which is corroborated by largely overlapping histograms of the longest bud lengths at day 7 (FIG. 4I). These results indicate that the initial concentration of growth factor/nutrients in proximity to the organoids influences early growth kinetics.

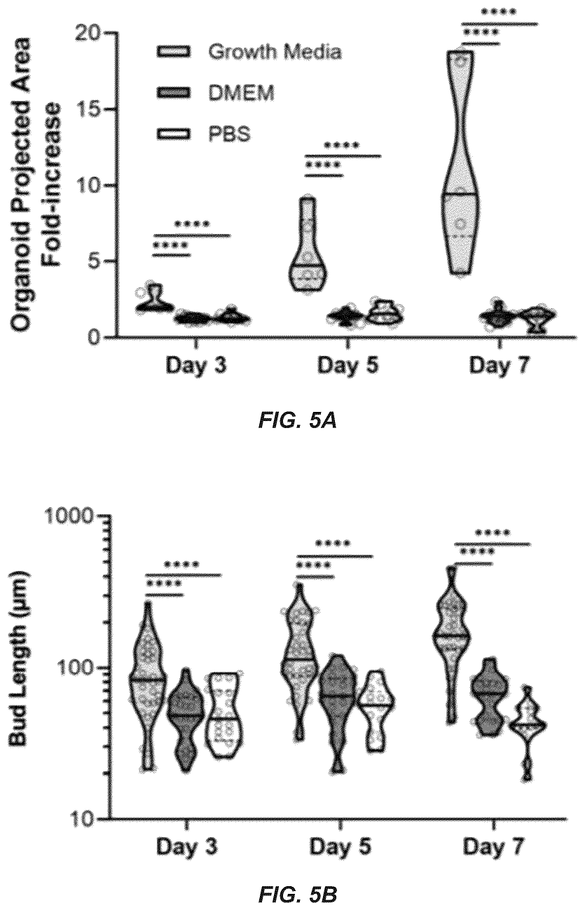

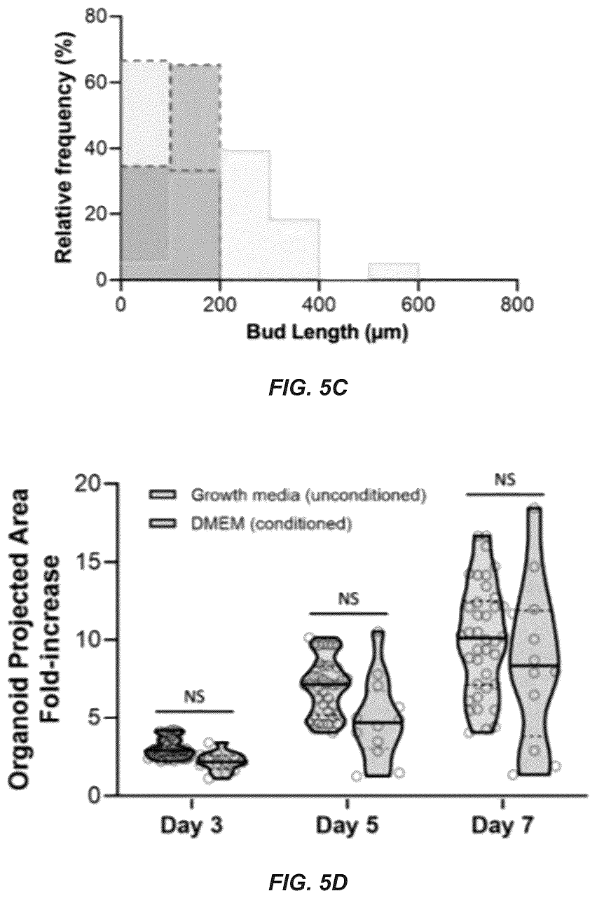

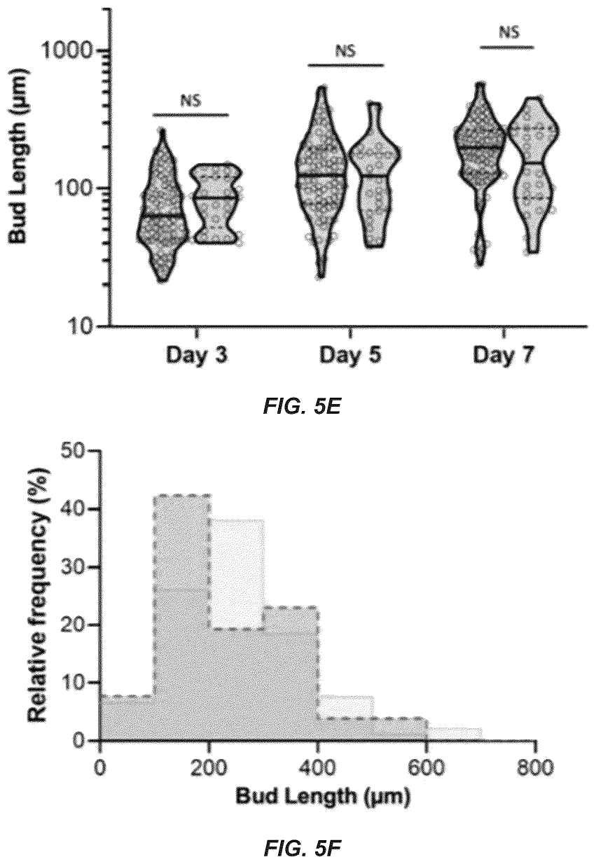

Next, experiments were devised where organoid growth media in the top compartment was swapped for DMEM/F12 and PBS, hypothesizing that the growth media on top was responsible for providing a sufficient amount of growth factors initially. Analysis of organoid projected area and longest bud length revealed that organoids grown in dual layers where the top compartment had DMEM/F12 or PBS instead of growth factor-containing media failed to grow altogether (FIG. 5A-5C), thus supporting the hypothesis. Strikingly, conditioning the agarose in dual layers where the top compartment is growth factor-free (i.e. DMEM/F12) provided full recovery of organoids, as exhibited by its parity with regular dual layer cultures (growth media on top, unconditioned agarose) (FIG. 5D-5F). Taken together, the initial concentration of growth factors is a key factor in successful organoid expansion in the Transwell dual layer setup, and that transport of growth factors from the bottom compartment alone through the agarose layer is capable of supporting organoid expansion for the remainder of culture since the top compartment remained untouched throughout the 7 or 13-day cultures.

### Bioprinting MATRIGEL-Agarose Core-Sheath Filaments

Having established how agarose complements MATRIGEL for intestinal organoid culture, we then established a protocol for bioprinting using these materials. Previous work demonstrated the limited printability of blended MATRIGEL-agarose composite bioinks (Fan, R. et al. J. Biomater. Appl. 2016 31:684-692). As such, a core-sheath filament strategy was decided in which an agarose layer surrounded an organoid-laden MATRIGEL core, thus providing the mechanical integrity of agarose while keeping the MATRIGEL layer intact (FIG. 6A). The cornerstone of this technique is the continuous extrusion and gelation of agarose within the nozzle through tight control over agarose temperature and flow rate, which are chosen to ensure ideal flow/extrusion behavior. Specifically, agarose is fed into the nozzle as a warm pre-gel liquid where it flows around the inner glass capillary. In the course of the flow through the nozzle, agarose cools and turns into a viscoelastic gel as it exits the nozzle, thus resulting in a relatively homogeneous and smooth agarose filament in contrast to the rough and chunky texture from our previous work. As the agarose filament emerges from the nozzle, an internal cavity/channel corresponding to the shape of the inner glass capillary is formed that is subsequently filled by flowing MATRIGEL bioink.

Agarose filaments produced through this method exhibit high optical transparency, display high shape fidelity and exhibit sufficient mechanical integrity for ease of handling. As seen in FIG. 6B, centimeter scale core-sheath filaments can be produced while cells suspended in the MATRIGEL core are capable of expanding and self-organizing to the point where it is visible to the naked eye. Inspecting the filament's cross section, we see that there is a defined core channel wherein organoid-laden MATRIGEL is located, and that this core-channel takes on the shape of the inner capillary, which in this case is an 800 μm×800 μm square. With sufficiently high cell seeding density, filaments can achieve nearly full coverage of the core volume and create continuous epithelial tubes. Compared to other reports of tissue engineering with agarose where agarose is first micropatterned before being loaded with MATRIGEL (Han, S. et al. Biophys. Res. Commun. 2018 496:785-791), our filament printing strategy is capable of producing MATRIGEL-agarose filaments in one step without the need to multiple pattern transferring steps.

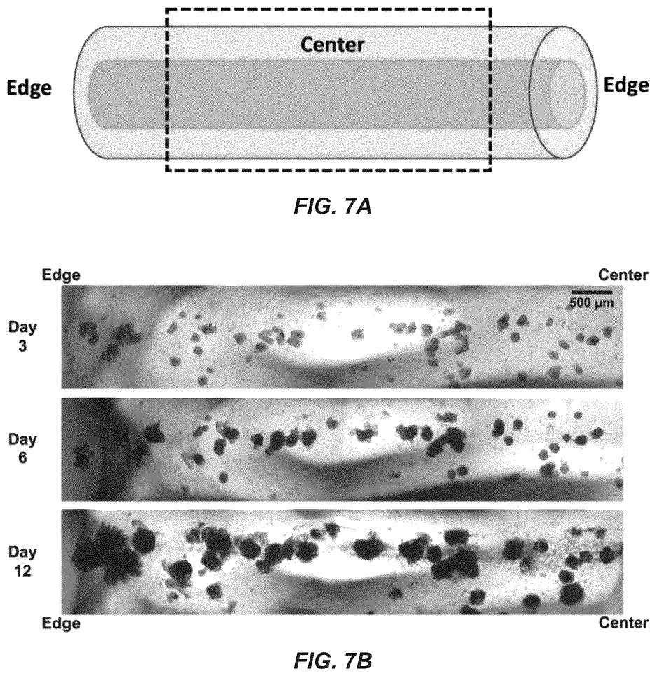

Throughout the duration of 12-day culture, the agarose sheath remained stable with no sign of mechanical damage or distortion, thus maintaining a consistent boundary/interface with the MATRIGEL core. Timelapse images of intestinal organoids revealed growth and expansion that is consistent with regular organoids in that budding structures form in addition to darkened core domains (FIG. 6C). The organoid growth in filaments was compared with different geometries (e.g. agarose sheath and MATRIGEL core thickness). Longest bud length data shows that thin MATRIGEL cores are more conducive to organoid growth than thicker cores, with 0.5 mm diameter cores producing median longest bud lengths that are 86% greater than in 1.5 mm diameters at day 12 (185.9 μm versus 97.1 μm) (FIG. 6D-6E). Indeed, close inspection of organoids in thick-core filaments reveals poorer growth at the center of the filaments away from the edges over the course of 12-day culture. Organoids located furthest away from the edge of the filament and from the agarose-MATRIGEL interface exhibit limited expansion and produce fewer pronounced budding structures compared to organoids at the edge (FIG. 7). Interestingly, the distribution of longest bud lengths in the bioprinted filaments, regardless of geometry/thickness, is markedly lower than those observed in the Transwell setup at similar time points. This was attributed to the difference in layout between the Transwell setup and the core-sheath filaments, with the former having two separate media compartments, one of which is immediately adjacent to the MATRIGEL layer containing the agarose.

### Dislodging MATRIGEL Cores From Core-Sheath Filaments

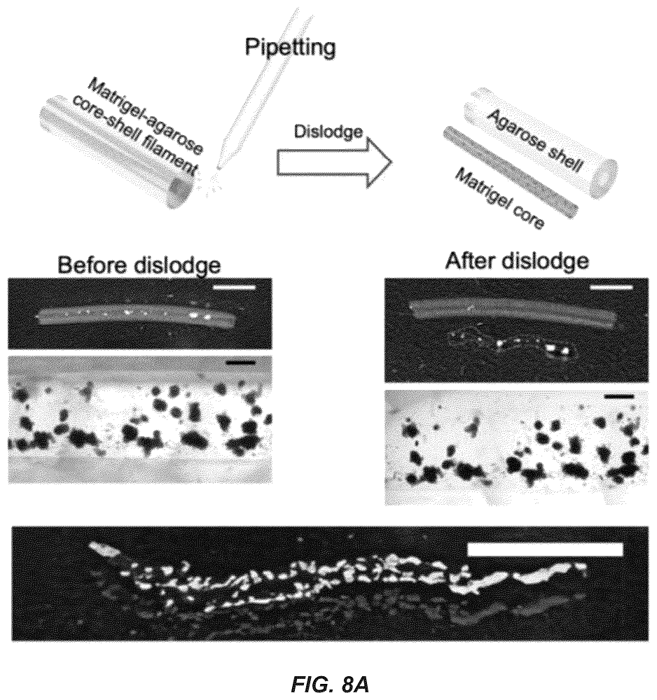

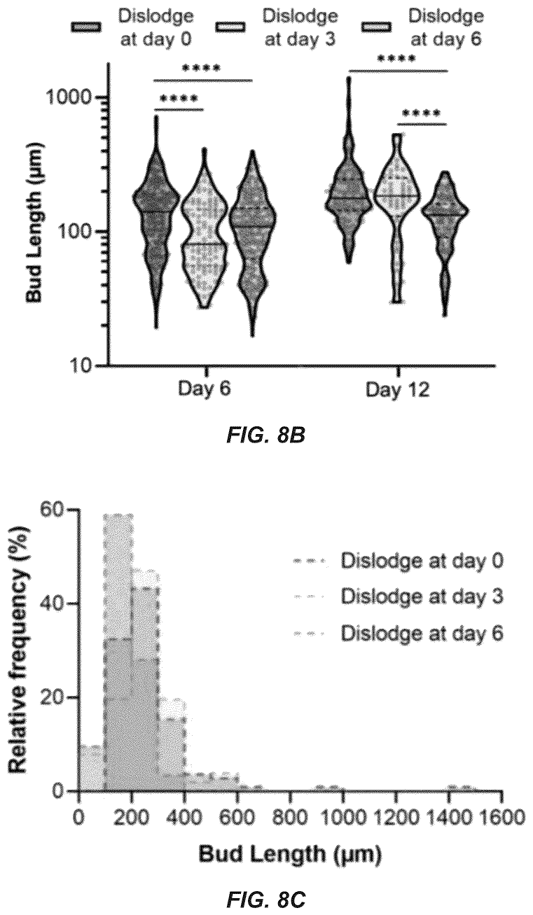

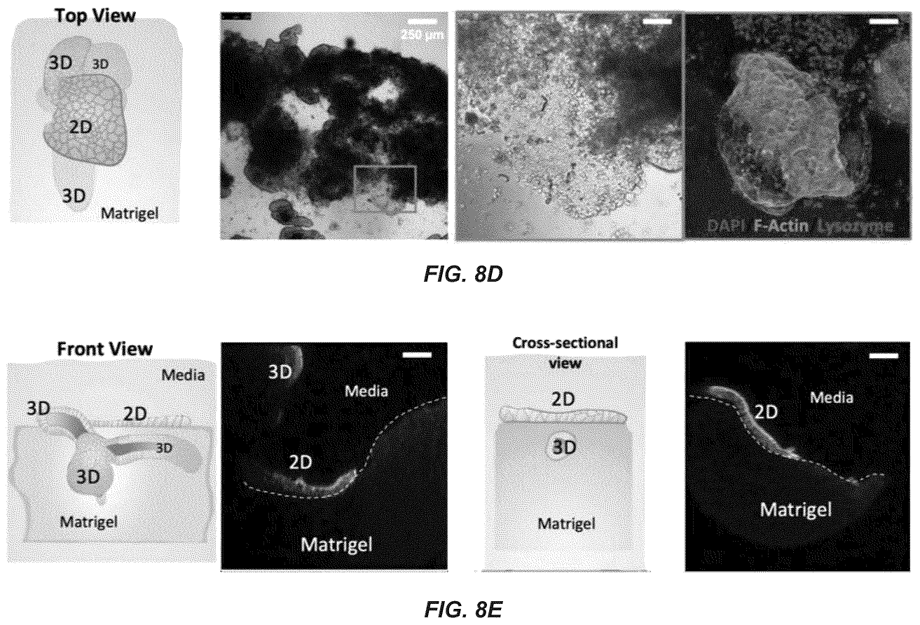

It was noticed during the preparation of Transwell dual layers that MATRIGEL adheres poorly onto an agarose surface and that light mechanical agitation is often sufficient to successfully displace MATRIGEL. The same is true in the MATRIGEL-agarose core-sheath filaments wherein MATRIGEL cores could be dislodged from the agarose sheath when subjected to sufficient agitation of the liquid media, whether intentionally or otherwise. Thus, in addition to producing core-sheath filaments, our bioprinting protocol is capable of producing thin MATRIGEL filaments (<1 mm diameter) that took on the shape of the inner glass capillary used during the printing process. Although dislodged filaments freely float and flex within the culture media, they roughly retain their original shape over the course of culture (FIG. 8A).

Analyzing the longest buds of organoids within dislodged filaments, it was observed that the time of dislodging plays a significant role in subsequent organoid expansion, with early times of dislodging (i.e. dislodge at day 0) resulting in the highest longest bud lengths (FIG. 8B-8C) and exhibiting a greater proportion of organoids with elongated budding structures (>500 μm) by day 12 of culture. Compared to the MATRIGEL-agarose core-sheath filaments which had 1% organoids exhibiting elongated budding structures, day 0 and day 3-dislodged filaments had 5.4% and 3.9 (FIG. 8C). These results confirm once more that the positive impact of agarose on organoid growth in the context of the Transwell setup did not translate over to the bioprinted context. This again was attribute to the absence of a separate media compartment adjacent to the organoid-laden MATRIGEL layer that is otherwise present in the Transwell setup.

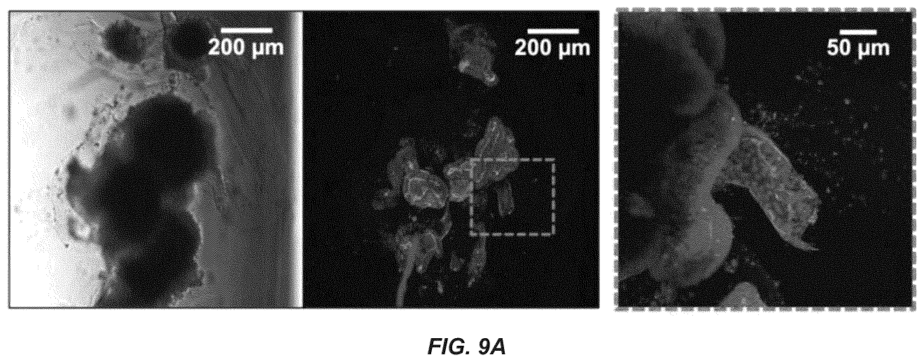

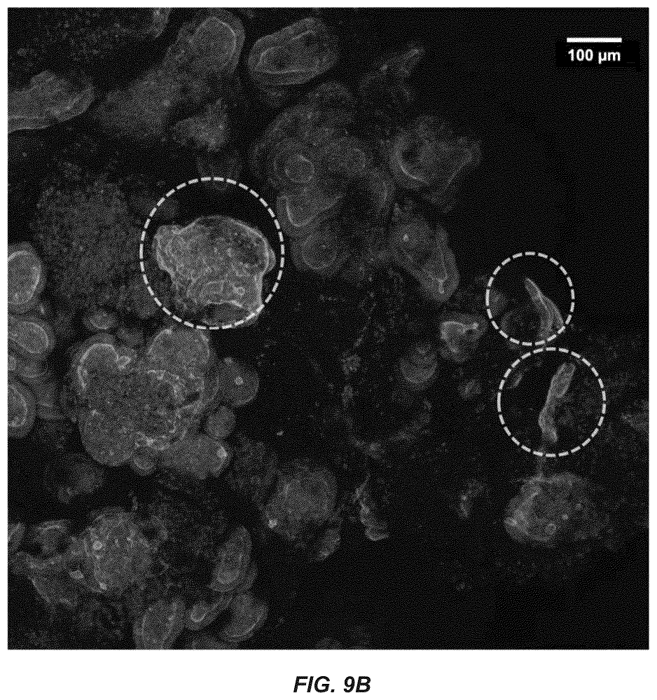

Strikingly, there were a number of occurrences where organoids from dislodged filaments developed hybrid 2D-3D intestinal epithelial morphologies at day 12 that deviated from typical organoid shapes. Specifically, these epithelial tissues exhibited surface monolayers, marked by their cobblestone-like appearance, alongside standard folding/budding structures regularly seen in intestinal organoids (FIG. 8D-8E). That these monolayers exist on the surfaces and not in the bulk MATRIGEL is confirmed by orthogonal views produced using Immunofluorescence microscopy also reveals high expression of F-actin in both apical and basolateral surface for certain regions of epithelial tissue, including but not limited to those corresponding to surface monolayers (FIG. 9). Since regular organoids grown in bulk MATRIGEL usually display F-actin on the luminal surface (i.e. the surface facing in), these regions of high F-actin on both surfaces would suggest a reorganization of epithelial cells and may be indicative of a transition from the folded/budding structure into a surface monolayer wherein epithelial cells are display apical-out polarity.

### Discussion

Agarose is known to be a bioinert polysaccharide and unable to support cell adhesion and growth. In terms of mechanics, agarose is an order of magnitude stiffer than MATRIGEL and has a higher crossover frequency (Zuidema, J. M., et al. J Biomed Mater Res B Appl Biomater. 2014 102(5):1063-73), allowing it to exhibit more gel-like/elastic behavior under the mechanical loading conditions associated with regular handling. As such, previous reports of using agarose in tissue engineering involve exploiting agarose's mechanical integrity and ease of molding to pattern physical barriers to guide neuron growth to promote axonal regeneration (Han, S. et al. Biophys. Res. Commun. 2018 496:785-791) or to create custom in vitro neuronal networks (Krumpholz, K. et al. ACS Appl. Mater. Interfaces 2015 7:18769-18777). For intestinal epithelial engineering, previous work with HCT116 cell lines grown in agarose-MATRIGEL composites display cell viability and spreading (Fan, R. et al. J. Biomater. Appl. 2016 31:684-692), but such has not been demonstrated for primary-derived intestinal epithelial cells. The results in the Transwell and bioprinted filament contexts demonstrate that bioinert hydrogels such as unmodified agarose are capable of being used in tandem with MATRIGEL to support in vitro intestinal epithelial growth once certain conditions are met, namely sufficient levels of growth factors pertinent to intestinal stem cell biology. This contrasts with a 2017 paper which reported that monolayers grown on agarose-collagen substrates did not exhibit any measure of self-renewal (Wang, Y. et al. Cmgh 2017 4:165-182.e7). This difference in outcome was attribute to variation in growth factor distribution, undefined composition of agarose and possible differences between culture requirements for intestinal 2D monolayers versus 3D organoids in vitro.

The occurrence of highly elongated budding structures (>500 μm in length) in Transwell cultures (and in dislodged filaments to a lesser extent) is likely the result of those particular budding structures exhibiting heightened proliferation rates that far surpass the rate at which new crypts can emerge. This is supported by computational simulation studies of multicellular structures that show how the occurrence/prevalence of tubulation/branching behavior depends on the relative time scales between patterning (e.g. cell signaling) and deformation (e.g. proliferation) (Okuda, S., et al. Sci. Rep. 2018 8:1-15). Mechanistically, researchers have long identified the Wnt signaling pathway as responsible for promoting proliferation in intestinal epithelium, and that gradients in Wnt signaling can result in tissues that are polarized into distinct proliferative and differentiated domains (Attayek, P. J. et al. PLoS One 2016 11:1-23; Libby, A. R. G. et al. Dev. 2021 148). It was hypothesized that aspects of the Transwell dual layer setup accentuates Wnt signaling in the transverse (i.e. in-plane direction) and results in hyperpolarization of the organoid, so long as there is a sufficient level of growth factors at the initiation of culture.

Previous research has shown the importance of removing Wnt signaling antagonists (e.g. Dkk-1) secreted by intestinal epithelial cells to enhance tissue organization. It has been noticed that organoid densities play a non-negligible role in subsequent growth performance, with higher densities often promoting poorer growth quality and smaller/shorter morphologies due to excess local Dkk-1 levels around the organoids (Shin, W., et al. iScience 2019 15:391-406; Shin, W. et al. iScience 2020 23:101372). In this Transwell dual layer setup, liquid media on the top compartment allows Wnt antagonists to diffuse out from the MATRIGEL layer and thus prevent buildup around the organoids. At the same time, there would be buildup of Wnt antagonists just underneath the organoids at the interface of the MATRIGEL and underlying agarose layers simply due to the relative difference in diffusion rates. This leaves only the tranverse directions along the MATRIGEL layer available for high levels of Wnt signaling and thus driving hyper-polarization and proliferation in the tranverse directions. Nevertheless, further studies on this elongation phenomenon should specifically interrogate the role played, if any, by Wnt antagonists such as Dkk-1 secreted by cells. At the same time, previous literature that documented the formation of elongated budding structures in intestinal organoids associated the elongated morphologies with a regenerative phenotype that emerges upon intestinal injury, and that YAP-signaling is heavily implicated in inducing and maintaining this phenotype (Qu, M. et al. Cell Res. 2021 31:259-271; Sprangers, J., et al. Cell Death Differ. 2021 28:95-107). Thus, targets associated with YAP-signaling should also be the focus of future mechanistic studies on the role of agarose in promoting intestinal growth and elongation.

Standard intestinal epithelial culture using primary-derived cells typically consist of 2D surface monolayers or 3D organoids. Drawbacks of the former include lack of physiological architecture (e.g. crypts and villi) whereas the latter lacks accessibility to the apical surface/compartment. The hybrid 2D-3D morphology observed in intestinal epithelial tissues from dislodged filaments presents and interesting possibility to break the tradeoff inherent in 2D monolayers or tissue cultures. Factors that may contribute to the occurrence of hybrid 2D-3D morphologies include occasional fluid shear mid-culture and the emergence of organoids beyond the MATRIGEL-media interface.

The development of core-sheath MATRIGEL-agarose filaments reported in this work is part of a larger trend in tissue engineering wherein multiple biomaterials are arranged hierarchically to improve tissue growth, uniformity, visibility or usability or all of the above (Mistry, P. et al. Macromol. Biosci. 2017 17:1-8; Tallapragada, N. P. et al. Cell Stem Cell 2021 28:1516-1532.e14). The core-sheath filament bioprinting approach disclosed herein has several key advantages in terms of reliability of filament quality (e.g. optical transparency, ease of handling) and workflow flexibility (e.g. customizable core-sheath geometry, ability to manipulate the MATRIGEL core post-gelation). While the lack of adhesion between agarose and MATRIGEL allowed for the selective formation of thin MATRIGEL filaments, further development in the agarose-MATRIGEL material system should consider methods for tuning the chemistry of agarose or other similarly mechanically robust hydrogels to render it more adhesive to the MATRIGEL, thus limiting the occurrence of unintentional dislodging and enabling more consistent/stable positioning of MATRIGEL relative to agarose.

### Conclusion & Outlook

In summary, agarose-MATRIGEL dual layers can be used for intestinal organoid growth and expansion, documenting not only agarose's compatibility intestinal organoids, but also its crucial role in inducing a highly elongated morphology. These findings raises new possibilities on how different biomaterials can engineered to interact with various morphogen signaling to thus offer spatiotemporal control over tissue architecture and cellular fate. Moreover, using a core-sheath layout, a bioprinting approach is presented that is flexible in its ability to either generate continuous core-sheath filaments or dislodged cores that support intestinal epithelial tissue growth in vitro. Overall, the approach presented herein is represents a continuing trend of engineering multiple hydrogel materials to improve the performance, capabilities or ease of experimentation in the area of tissue engineering and bioprinting.

### Example 2

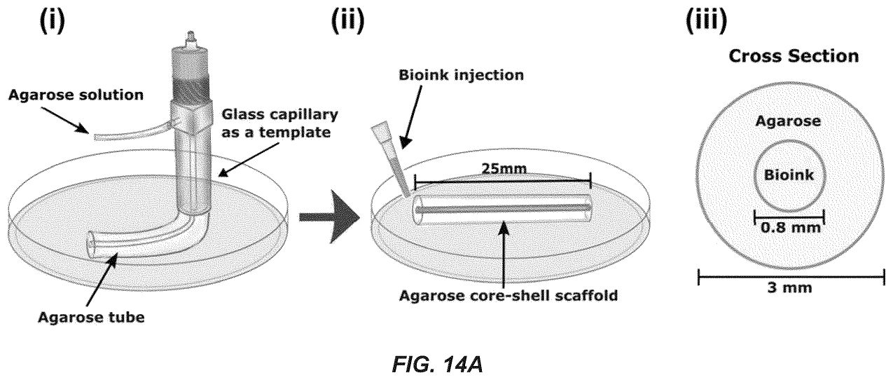

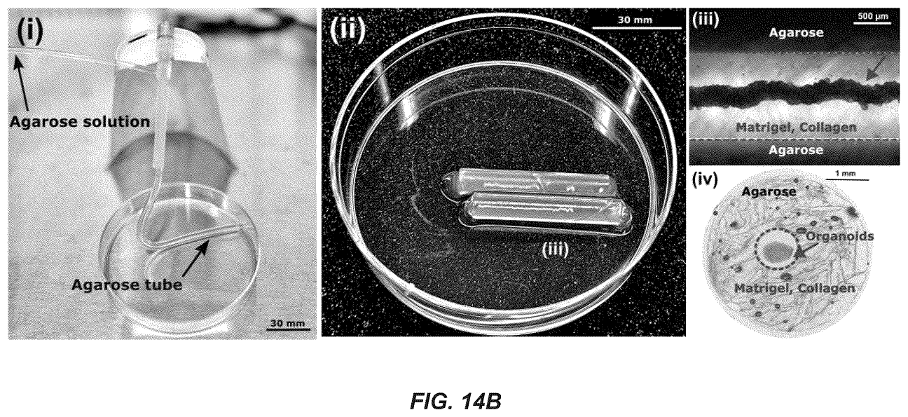

FIGS. 14A and 14B show bioprinting agarose core-shell scaffold for MCF10A culture. FIG. 14A contains schematics of (i) printing agarose tube using a customized printhead nozzle and a glass capillary, (ii) injecting cell-containing bioink to printed agarose tube, (iii) a cross-section of bioink-filled agarose filament. FIG. 14B contains experimental images of (i) printed agarose tube, (ii) bioink filled agarose filaments, (iii) a zoom-in microscopic image of a bioink filled agarose filamen and (iv) its cross-sectional image. Dotted blue circle indicates the bioink and red arrow indicates MCF10A organoids. Bioink contains 40% (v/v) Matrigel and 10% (v/v) collagen. Images of ii-iv in B were taken after 4 days of culture in growth media.

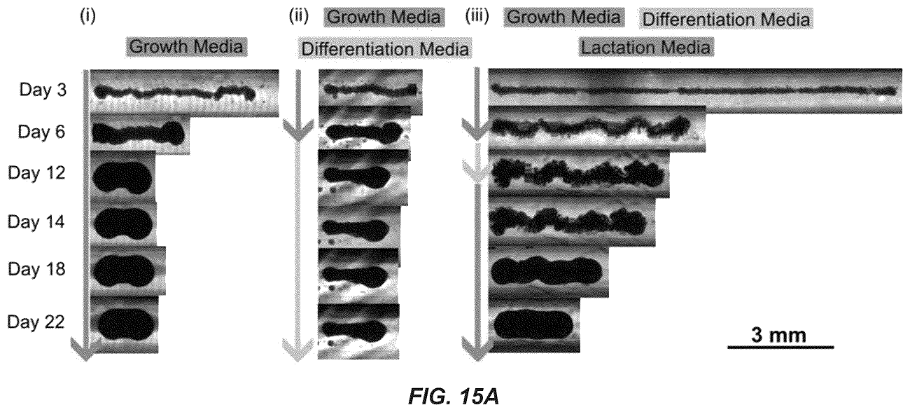

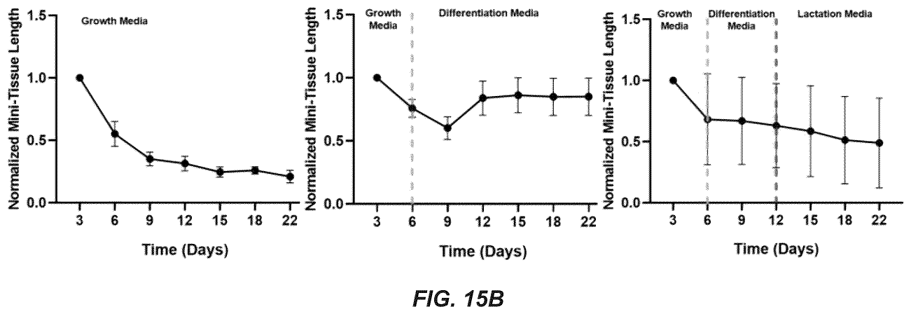

FIGS. 15A and 15B show agarose core-shell scaffold promotes the growth of MCF10A into dense, connected mammary organoids. FIG. 15A contains brightfield images of the growth of MCF10A cells in (i) growth media only, (ii) growth media and differentiation media, and (iii) growth media, differentiation media and lactation media. Note differentiation media was added at day 6 in (ii) and lactation media was added at day 12 in (iii). FIG. 15B contains plots of normalized filament length over time. n=3, 6, and 14 filaments for growth media, growth and differentiation media, and growth, differentiation and lactation media, respectively. Dotted vertical lines dictate the day to change media type. Data are represented as mean±SD.

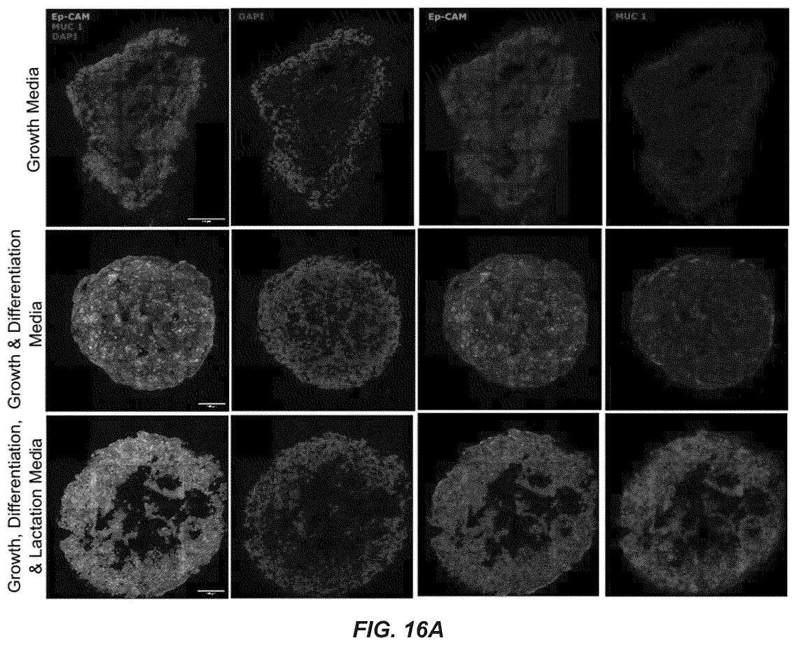

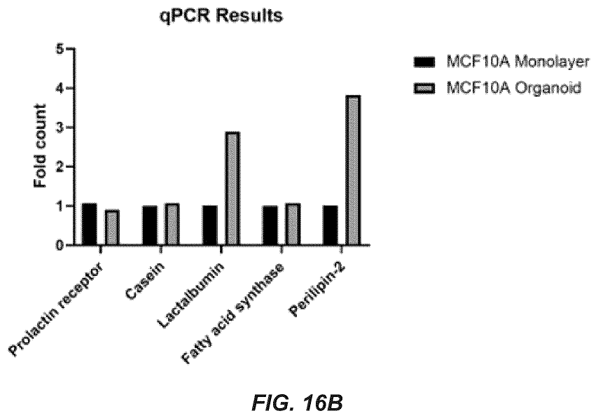

FIGS. 16A and 16B show laction media enhances the expression of epithelial mucin (MUC 1) in MCF10A organoids. FIG. 16A contains confocal images of the cross section of MCF10A organoids cultured in growth media only, growth and differentiation media, and growth, differentiation, and lactation at day 22 (left to right). DAPI stains for nucleus, MUC-1 stains for the mucin protein, Ep-CAM is an epithelial cell adhesion molecule for cell-cell contact. FIG. 16B contains qCR results.

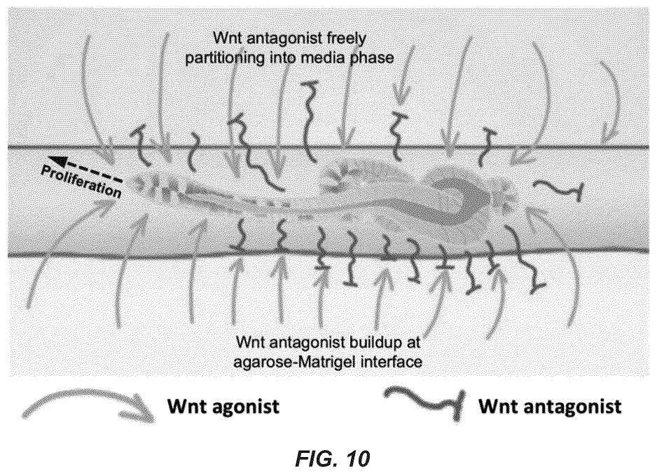

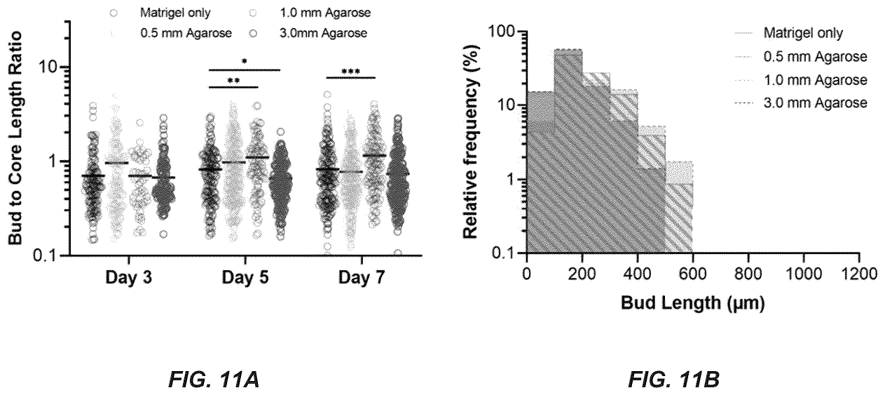

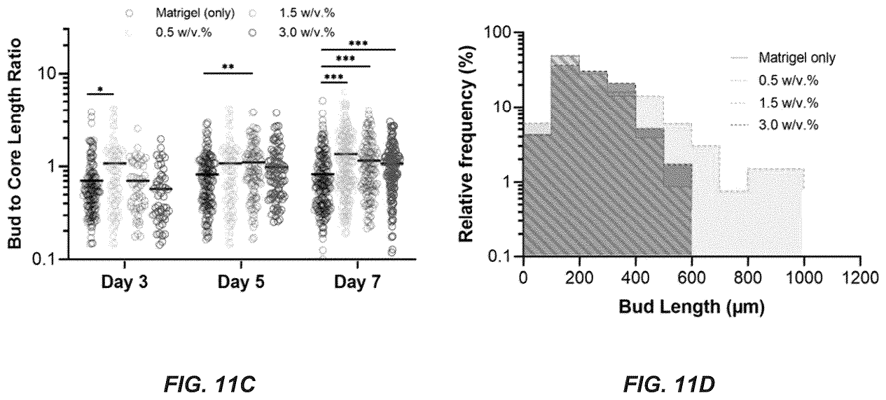

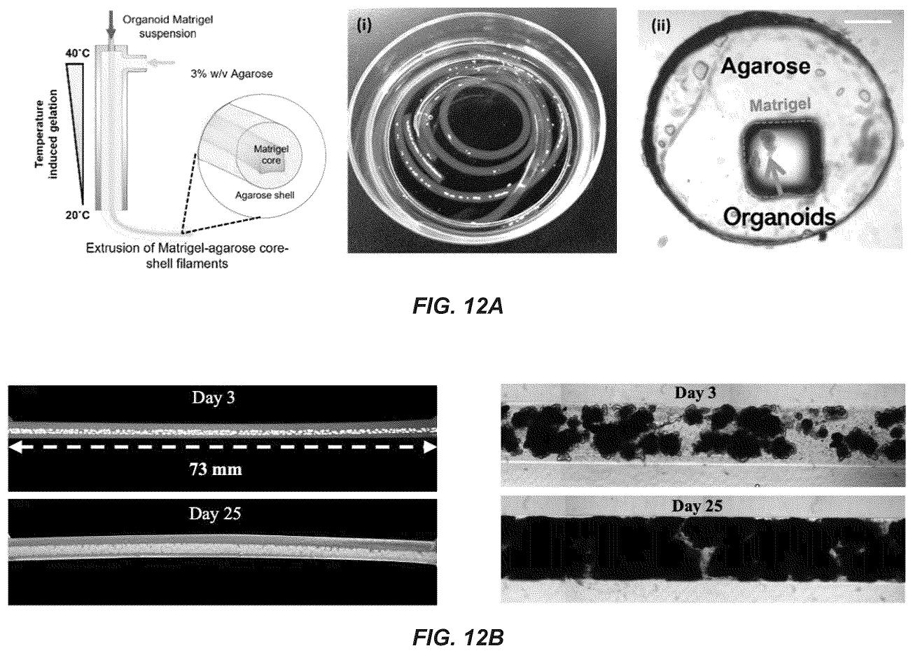

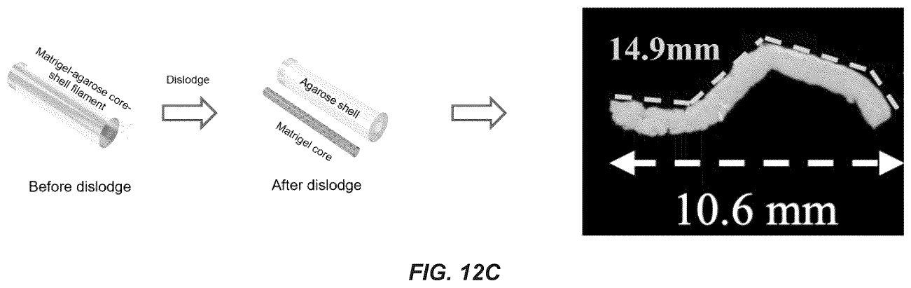

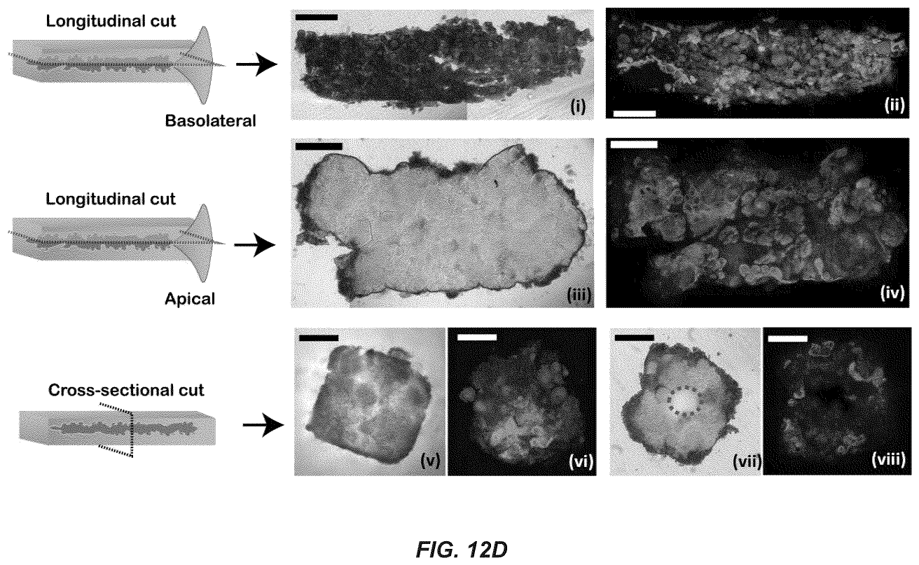

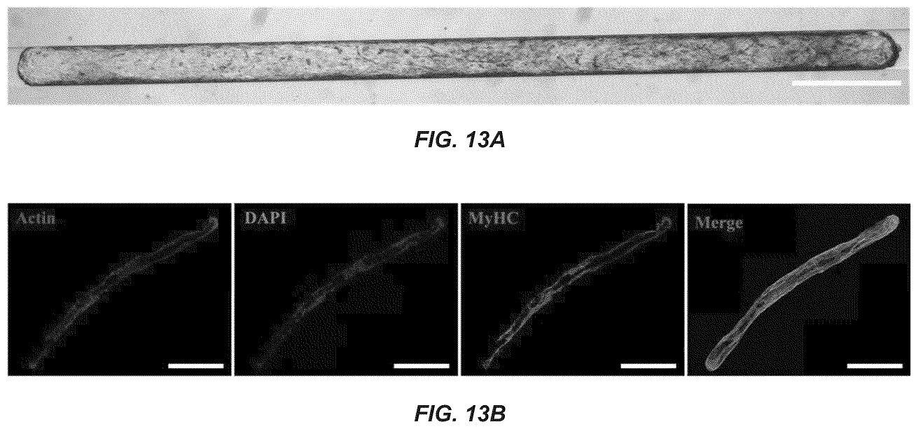

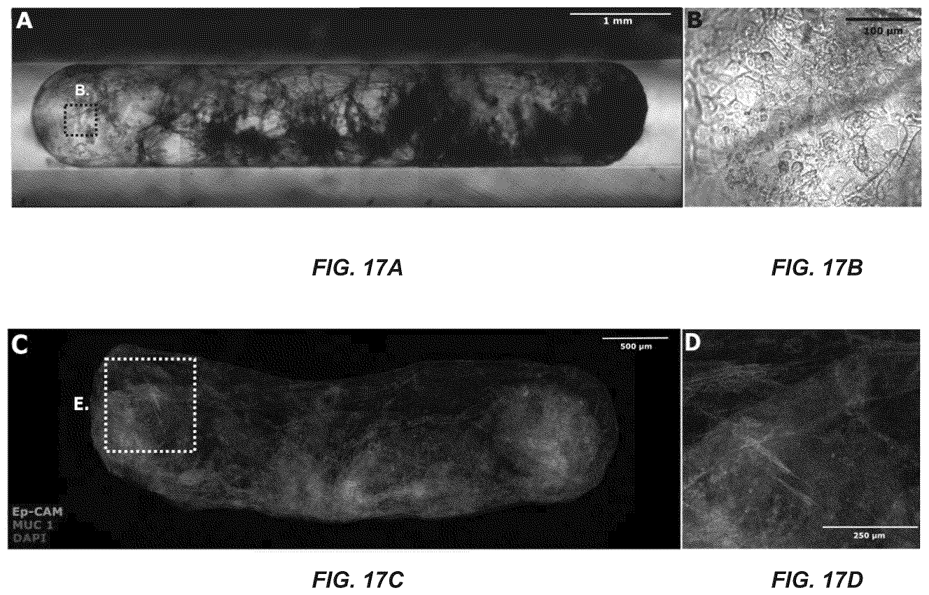

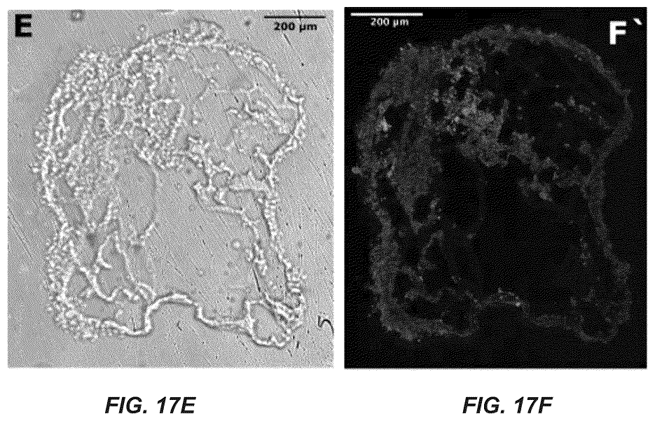

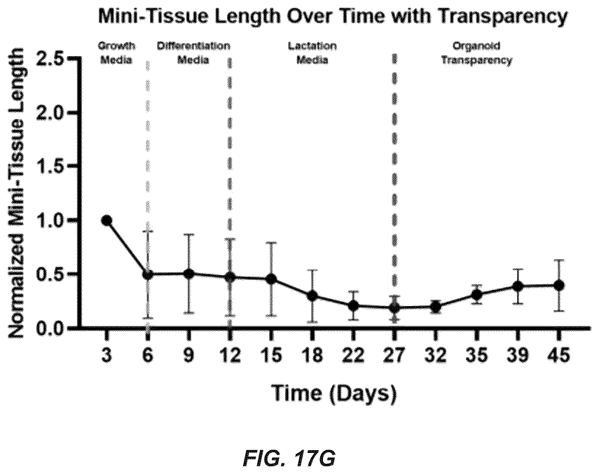

FIGS. 17A to 17G show long-term culture creates transparent, hollow MCF10A organoids. FIGS. 17A and 17B contain brightfield images of the transparent MCF10A organoids cultured at 32 days at 5× magnification (FIGS. 17A) and 10× magnification (FIG. 17B), revealing a monolayer cell network on the organoid surface. FIGS. 17C and 17D contain confocal images of transparent mini-tissue stained with Ep-CAM, MUC-1, and DAPI at 63× magnification of the entire tissue (FIG. 17C) and its zoom image (FIG. 17D). FIGS. 17E and 17F show cross-sectional bright field (FIG. 17E) and confocal images (FIG. 17F) of the hollow MCF10A organoids. FIG. 17G shows organoid length over time for mini-tissues that were seeded as a monolayer with Matrigel and collagen into agarose, resulting in mini-tissue transparency at day 27.

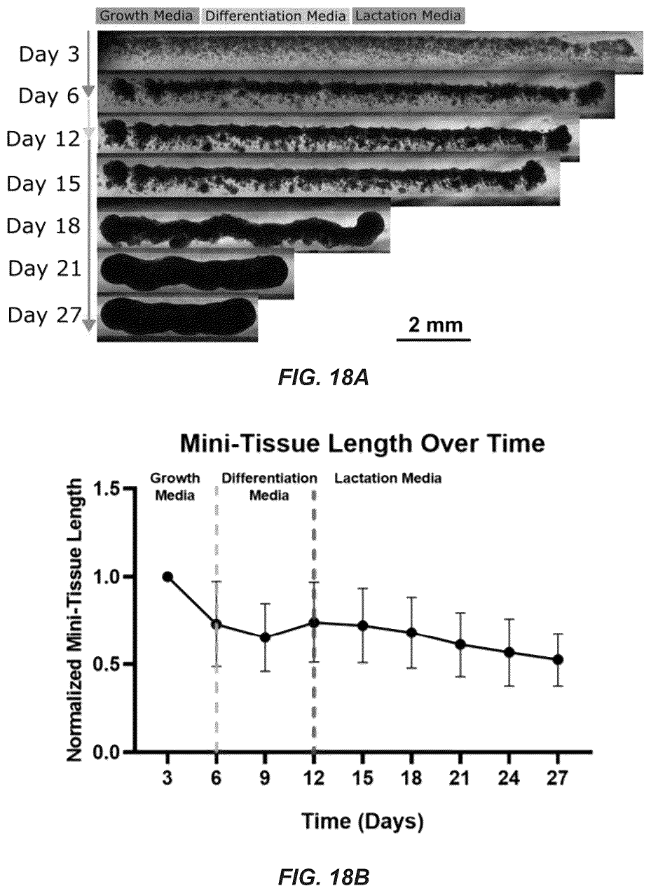

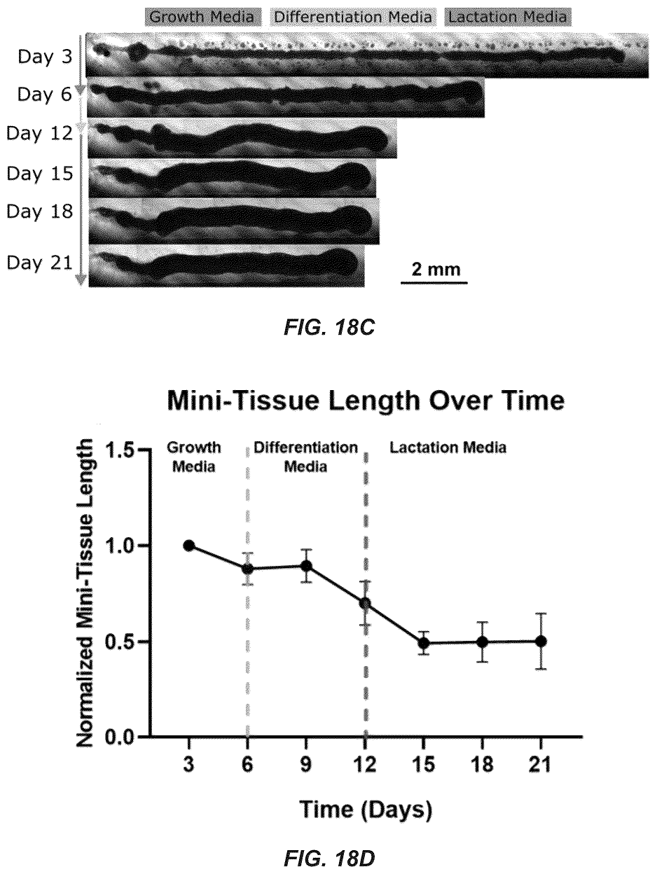

FIGS. 18A to 18D show MCF10A cells grown with Matrigel into organoids, then seeded into agarose filaments with Matrigel and collagen. Organoids were cultured in the agarose filaments in growth media until Day 6, cultured in differentiation media until Day 12, and lactation media until Day 22 (FIG. 18A). FIG. 18B shows measured mini-tissue length over time. Organoids cultured with pure Matrigel cultured with all media types. MCF10A cells seeded with only Matrigel formed mini-tissues which aggregated on Day 3 and shrank in length over time until day 21 (FIG. 18C). FIG. 18D shows mini-tissue length over time of this culture grown with Matrigel only. No difference in mini-tissue growth and shrinkage between a bioink with pure Matrigel and a bioink with a mixture of collagen and Matrigel.

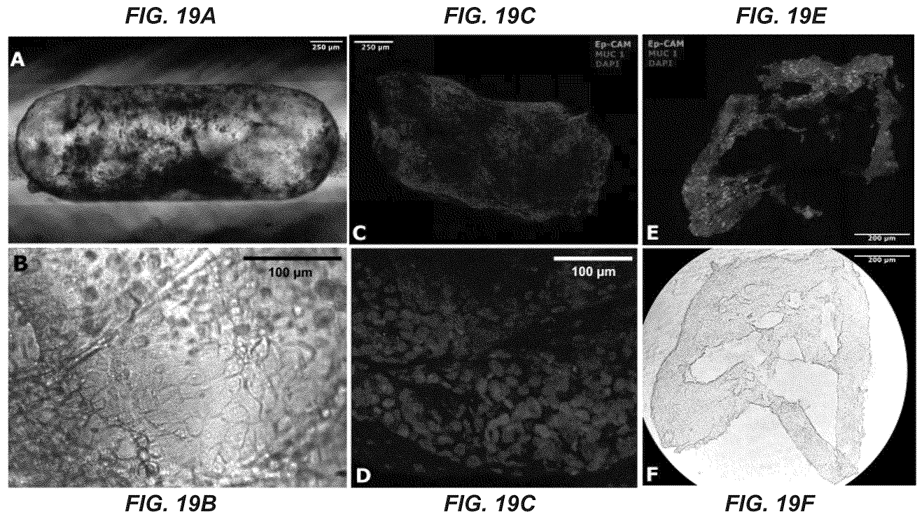

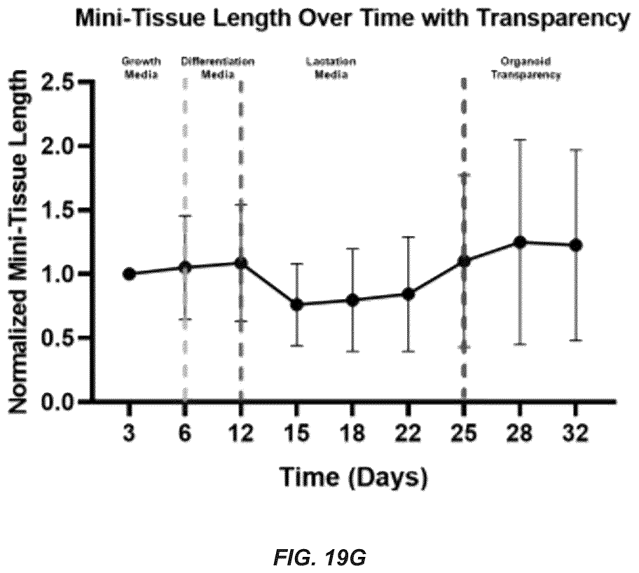

FIGS. 19A to 19G show transparent organoids grown by first culturing MCF10A cells in Matrigel for four days. Brightfield image of transparent organoid inside agarose at day 25 (FIG. 19A) and zoomed in to its monolayer surface (FIG. 19B). FIGS. 19C and 19D show confocal image of the same transparent organoid outside of agarose at 25 days (FIG. 19C) and zoomed in of the surface (FIG. 19D) Confocal images represent 100 stacked images. FIGS. 19E and 19F show confocal image (FIG. 19E) and brightfield (FIG. 19F) of cross section of transparent organoid at day 35. Confocal images represent 24 stacked images. FIG. 19G shows organoid length over time for mini-tissues that grew in Matrigel for 3 days prior to being seeded with Matrigel and collagen into agarose, resulting in mini-tissue transparency at day 25.

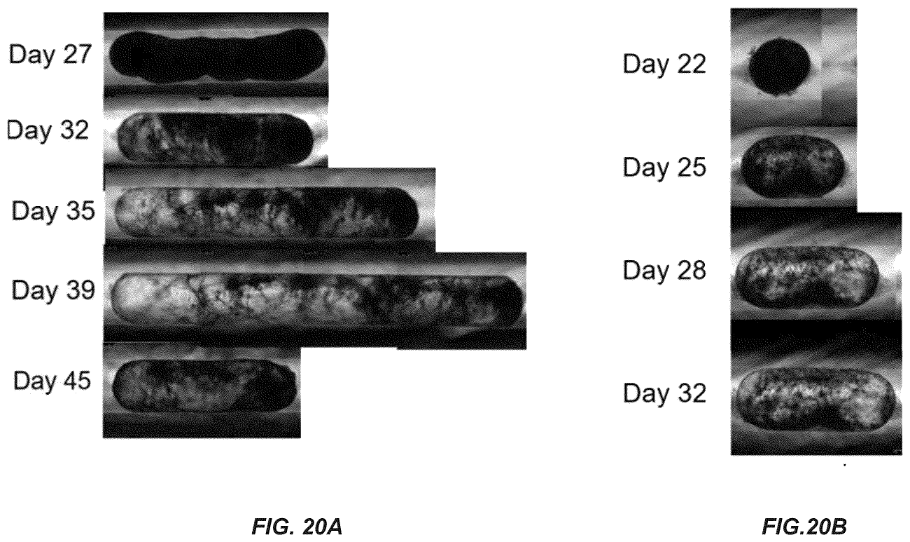

FIGS. 20A and 20B show timeline of transparent mini-tissues. Mini-tissue transparency is observed at Day 32 for cultures grown from MCF10A monolayer (FIG. 20A). Mini-tissue transparency is observed at Day 25 for cultures grown from MCF10A organoids cultured in Matrigel for 4 days (FIG. 20B).

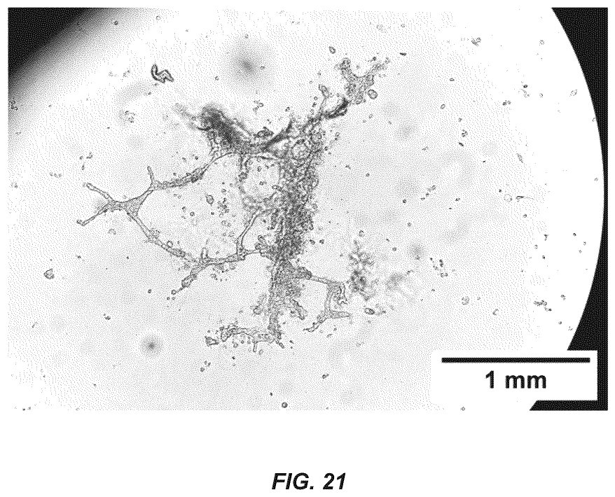

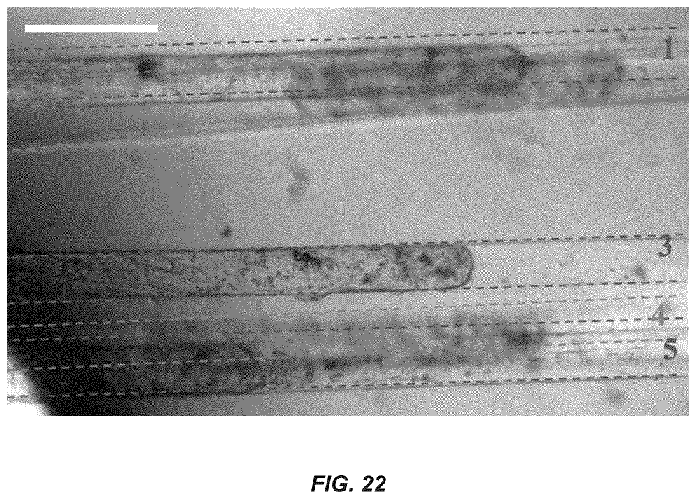

FIG. 21 is a brightfield image of branched structure from popped organoid. This branching structure was one of the leaked contents of the transparent organoid after prodding the transparent organoid with a glass pulled needle. The branch is reminiscent of the tubular structures found in mammary glands, confirmed from other literature.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A 3D organoid culture system comprising a filament having a bioactive hydrogel core covered by an inert hydrogel sheath, wherein the bioactive hydrogel core is an extracellular matrix hydrogel, and wherein the inert hydrogel has a diffusion rate for growth factors of from 1×10⁻⁷ to 9×10⁻⁷ cm²/s.

2. The system of claim 1, wherein the extracellular matrix hydrogel is MATRIGEL or collagen.

3. The system of claim 2, wherein the extracellular matrix hydrogel is a mixture of MATRIGEL and collagen.

4. The system of claim 3, wherein the extracellular matrix hydrogel comprises 90-70% (wt/v) MATRIGEL and 10-30% (wt/v) collagen.

5. The system of claim 1, wherein the inert hydrogel is polysaccharide hydrogel.

6. The system of claim 5, wherein the inert hydrogel comprises agarose or alginate.

7. The system of claim 1, wherein the core has a substantially cylindrical or cuboidal shape.

8. The system of claim 1, wherein the sheath has a substantially cylindrical or cuboidal shape.

9. The system of claim 1, wherein the core has an average diameter or width of from 0.3 to 1.0 mm.

10. The system of claim 1, wherein the sheath has an average thickness of from 2.0 to 6.0 mm.

11. The system of claim 1, wherein the bioactive hydrogel core does not comprise more than 0.1% (wt/v) agarose.

12. The system of claim 11, wherein the bioactive hydrogel core does not comprise agarose.

13. The system of claim 1, wherein the filament comprises two or more bioactive hydrogel cores covered by the inert hydrogel sheath.

14. The system of claim 1, further comprising stem cell organoids in the bioactive hydrogel core.

15. A method for producing an elongated epithelial tube from stem cell organoids comprising
(a) providing the system of claim 14; and
(b) culturing the system in a growth medium under growth conditions to produce the elongated epithelial tube.

16. The method of claim 15, wherein the stem cell organoid is an intestinal organoid and wherein the growth medium is an intestinal organoid growth medium.

17. The method of claim 15, further comprising removing the core containing the elongated epithelial tube from the extracellular matrix hydrogel.

18. An elongated epithelial tube produced by the method of claim 15.

19. A screening method comprising contacting the elongated epithelial tube of claim 18 with a candidate agent, culturing the elongated epithelial tube in a growth medium under growth conditions, and evaluating the elongated epithelial tube for a physiological effect.

20. The method of claim 19, wherein the physiological effect comprises one or more biomarkers of differentiation, one or more stem cell biomarkers, one or more gene mutations that lead to inflammatory bowel disease or colon cancer, or any combination thereof.
